(19) United States
(12) Patent Application Publication
(10) Pub. No.: US 2005/0009924 A1
(43) Pub. Date: Jan. 13, 2005

(54) Synthesis and pharmaceuticals of novel bis-substituted anthraquinone derivatives

(76) Inventor: Hsu-Shan Huang, Taipei (TW)

Correspondence Address: STEPHEN M. NIPPER;DYKAS, SHAVER & NIPPER, LLP, P.O. BOX 877, Boise, ID, 83107-0877, US

(21) Appl. No.: 10/615,695

(22) Filed: Jul. 8, 2003

(51) Int. Cl. A61K 31/137; A61K 31/12

(52) U.S. Cl. 514/649; 514/680; 552/244

(57) ABSTRACT

This invention relates to novel anthraquinone compounds useful in the treatment of allergic, inflammatory conditions, antioxidant, tumor condition, stem cell application, tissue engineering, applied in treating age-associate tissue degeneration, reverse organ failure in chronic high-turnover disease and therapeutic compositions containing such compounds. The compounds of the present invention are 1,4-, 1,5- and 1,8-difunctionalized anthraquinones or analogs thereof. According to the practice of the invention, there are provided bis-symmetrical substituted anthraquinone compounds according to formula I: 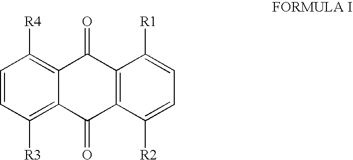
wherein R1, R2, R3 and R4 present a straight, aminoalkylamino side chains or branched chain alkyl group having 1 to 6 carbons which may be substituted with one or more groups of R5, or R1, R2, R3 and R4 present phenyl or benzyl which may be substituted with one or two groups of R6; wherein R5 is selected from the group consisting of halogen, —RNH₂, —RNH₂R, —ROH, —NO₂, —OCH₃, —OCH₂CH₃, and —OCH₂CH₂CH₃; and wherein R6 is selected from the group consisting of a straight or branched chain alkyl group having 1 to 4 carbons, halogen, —RNH₂, —RNH₂R, —ROH, —NO₂, —OCH₃, —OCH₂CH₃, —OCH₂CH₂CH₃, —CH₂Br, —CH₂Cl, —CH₂OH, —C(CH₃)₃, —(CH₂)₂0H, —(CH₂)₃OH, —(CH₂)₄OH, —CH₂NH₂, —(CH₂)₂NH₂, —(CH₂)₃NH₂, —(CH₂)₄NH₂, —(CH₂)₅NH₂, —CH₂N(CH₃)₂, —(CH₂)₂N(CH₃)₂, —(CH₂)₂NH(CH₂)₂OH, —(CH₂)₃NH(CH₂)₂OH, —(CH₂)₂NHCH₂OH, —(CH₂)₃NHCH₂OH, —CH₂CH(CH₃)₂, —CHCl₂, —CH(CH₃)Cl, —(CH₂)₂Cl, —(CH₂)₃Cl, —(CH₂)₃Br, —(CH₂)₄Br, and —(CH₂)₄Cl.

Chart 1. Activation of hTERT promoter-driven SEAP expression by c-Myc. About 1×10⁷ hTERT-BJ1 cells were transfected with 13.5 μg each of plasmid pSEAP or pPhTERT-SEAP and of plasmid pMT2T or pMT2T-cMyc by electroporation. After 24 h, viable cells were harvested, and reinoculated at a density of 3×10⁵/mL, and the SEAP activity after 24 h at 37 □. The transfection efficiency of each experiment was determined by cotransfection with 1.5 μg of plasmid pCMVβ. The values were determined from three experiments. P<0.05 is presented by an asterisk.

### BACKGROUND OF THE INVENTION

1. Field of the Invention

This invention relates to novel anthraquinone compounds useful in the treatment of allergic, inflammatory conditions, tumor condition, stem cell application, tissue engineering and therapeutic compositions containing such compounds. As a part of our program aimed at exploring the biological activity of symmetrical substitution of side chains into the anthraquinone chromophore, we have synthesized a series of 1,5-bisthioanthraquinones, 1,5-bisacyloxyanthraquinones, 1,5-bisaminoanthraquinones, 1,8-bisaminoanthraquinones, 1,4-bisamidoanthraquinones, and 1,5-bisamido anthraquinones that are related to the antitumor agent mitoxantrone. Since the telomerase-enzyme is a novel target for potential anticancer therapy and stem cell expansion, we also explore the biological effects of these compounds by evaluating their effects on telomerase activity and telomerase expression. These anthraquinone compounds possess antitumor, antiproliferative, antipsoriatic, anti-inflammatory, human telomerase activity, stern cell research, tissue engineering, or antioxidant activity.

2. Description of the Prior Art

A number of analogs of anthraquinone have been synthesized and evaluated both for preclinical antitumor efficacy as well as biochemical pharmacology. Morier-Teissier E. et al., J. Med. Chem., 36, 2084-2090 (1993). Anthraquinone-based compounds currently occupy a prominent position in cancer chemotherapy, with the naturally occurring aminoglycoside anthracycline doxorubicin and aminoanthraquinone mitdxantrone both being in clinical use. Anthraquinone derivatives display potent and selective antitumor activity, but their mechanism of action is not clearly established yet. Intercalating agents continue to occupy a prominent position in the treatment of malignant diseases and thus the antitumor and biochemical effects of these compounds remain as subjects of intensive research. The anthraquinone, mitoxantrone has been shown to have outstanding antitumor activities but a much narrower spectrum of activity in comparison with those of the anthracyclines. Krapcho A. P. et al., J. Med. Chem., 33, 2651-2655 (1990).

Its planarity allows an intercalation between base pairs of DNA in the conformation, while its redox properties are linked to the production of radical species in biological systems. Gatto, B. et al., J. Med. Chem., 39, 3114-3122 (1996). Despite structural similarities between the substitutents anthraquinone nucleus and molecules possessing known anti tumor activity, antiproliferative, antipsoriatic, antiinflammatory, human telomerase activity, stem cell research, tissue engineering, or antioxidant activity, these agents form a distinct mechanictic class. Perry P. J., et al., J. Med. Chem., 41, 3253-3260, 4873-4884 (1998); Perry P. J., et al., J. Med. Chem., 42, 2679-2684 (1999). Anthraquinone derivatives have been the subject of extensive research mainly due to their well-recognized biological importance and the significant biological applications. Although potential drug targets only present in cancerous cells have surfaced, the design of a drug which is selectively toxic to a tumor and not to the host organism is still very difficult have reported by Krapcho A. P., et al., J. Med. Chem., 41, 5429-5444 (1998).

Telomerase is required for telomere maintenance and is active in most human cancers and in germinal cells but not in most of the normal human somatic tissues. To facilitate the analysis of the expression of telomerase, we established in cancer and normal cell lines that carry secreted alkaline phosphatase (SEAP) gene under the control of hTERT. The effects of these compounds on the expression of telomerease were analyzed using the cell-based reporter systems. The effects of some of these compounds on hTERT expression appear to be specific because they did not increase the expression of a CMV promoter-driven SEAP. Thus, in addition to anticancer functions, our finding raises the possibility that these compounds might also have a role in cell immortilization. The application of these anthraquinone derivatives in stem cell research and tissue engineering is also discussed.

The chemical and biological activity exhibited by anthraquinone compounds is greatly affected by the different substituents of the planar ring system. As in the case of the anthracyclines, the mechanisms by which the antitumor anthraquinones kill cells are poorly understood and probably multimodal in their nature. It appears that the relative location of the planar and side-chain groups plays a major role in affecting enzyme function and sequence specificity. The significant clinical activity of mitoxantrone makes the development of second-generation anthraquinone congeners having better therapeutic efficacy together with reduced side effects an attractive area of investigation. The mode of action of anthraquinone leads to the conclusion that no single mechanism is predominantly operative and oxygen radicals play a crucial role in the proinflammatory action. As noted above, cancer is typically characterized by hyperproliferative component. There is thus a continuing need for effective compounds that address these aspects of cancer disease. To gain a wider understanding of the involvement of radicals in the action of anthraquinone-derived agents, several related compounds bearing selected characteristic functional groups were designed. The approach was to develop structure-activity relationships (SARs) of anthraquinone analogs with redox-active centers attached to the anthraquinone skeleton through spacer side chains at position 1, 4, 5 and 8, together with substituents with DNA-binding affinity. This invention describes the design and synthesis of anthraquinone that incorporate in their structure a potential antioxidant component and the results of relevant biologic studies.

We have recently started to explore the changes in the physicochemical and biological properties of unsymmetrical substituted anthracenes and symmetrical substituted anthraquinones, which exhibit unsymmetrical and symmetrical bis-substituted C—O—C and C—S—C linkage between the planar ring system and the side-chain substituents. Structure-activity relationship studies led to the discovery of symmetrical thio-substituted anthraquinones, which showed potent cytotoxicity with in terms of mean IC₅₀ value in cultured tumor cells and lipid peroxidation, respectively. Anthracyclines and anthraquinones form ternary complexes with DNA and the enzyme and stimulate DNA cleavage in a sequence-specific manner. Consequently, a considerable effort has been put forth to develop structural analogs or other classes of DNA binders which may circumvent or at least reduce disadvantage. Based on these facts, there are still demands of synthesis of new analogs of anthraquinone to provide good antitumor activities and less toxic side effects. On the other hand, managements of antitumor therapy are necessarily complementary which antioxidation is one of the most interesting topics investigated. This has enabled us to address the issue of how, and to what extent, the position of side chain substituents affects biological activity.

Since some of the compounds retained remarkable biological activity, this class appears to be worthy of further examination. We have previously shown that 9-substituted 1,5-dichloroanthracenes at U.S. Pat. No. 6,369,246 B2 (2002), 9-substituted 1,8-dichloroanthracenes at U.S. Pat. No. 6,372,785 B1 (2002) and 10-substituted 1,5-dichloro-9(10H)-anthracenones at U.S. Patent (2003); In these previous patents, we described the synthesis, biological evaluation and structure-activity relationships for anthracenes derivatives. We also reported a convenient synthetic pathway that leads to symmetrically substituted 1,5-bisthioanthraquinones and 1,5-bisacyloxyanthraquinone derivatives (Huang H.-S. et al., Arch. Pharm., 10, 481-486 (2002); Huang H.-S. et al., Chem. Pharm. Bull., 50(11), 1491-1494 (2002)) and at POC patent which application no. was 02-144700.4 on 2002/12/04 and ROC patent application no. was 091120375 on 2002/09/02. In order to provide further insight into anthracene and anthraquinone pharmacophore, the involvement of free radicals, anti-inflammatory, antioxidant, stein cell research, tissue engineering and antiproliferative activity, we examined the effects of introducing symmetrical electron-donating 1,5-bisoxy, 1,5-bissulfur, 1,5-bisamino, 1,8-bisamino, 1,4-bisamido and 1,5-bisamido di-functionalized anthraquinone derivatives to see where replacement of the electron-withdrawing or electron-donating groups of the series compounds can provide analogs with potential biological activities. Despite the extensive and long-standing therapeutic utilization of anthraquinones, their mechanism of action is still uncertain. The positional attachment of the side chain has been shown to profoundly influence their activity. Therefore, the need still exists for anthraquinone or anthracene congeners endowed with improved therapeutic efficacy and less toxic side effects, as well as effectiveness against multiple drug-resistant (MDR) cell lines

### SUMMARY OF THE INVENTION

The present invention relates to novel symmetrical bis-substituents anthraquinone compounds, and analogs thereof having therapeutic utility with respect to tumor conditions, allergic, inflammatory conditions, antioxidant activity, stem cell research, tissue engineering and therapeutic compositions containing such compounds. In particular, many of the improved anthraquinone compounds provided for according to the practice of the invention are effective at low concentrations for treatment of patients suffering from tumor conditions and all of therapeutic compositions containing such compounds. Because these compounds may be administered at low concentrations, the undesirable allergic or inflammatory effects caused, in whole or in part, by free radicals or active oxygen species that are generated by anthraquinone compounds are substantially eliminated. Accordingly, in one embodiment of the invention, there is provided an anthraquinone compound according to formula I as defined below and shown in below, said compound containing substituent R1, R2, P, and R4. The substituent R, wherein R represents a branched or straight chain alkyl group having from 1 to 6 carbon atoms, said alkyl group being substituted with at least one substituent selected from the group consisting of difunctionalized amino, amido, acyloxy, thio, substituted phenyl, benzyl and substituted benzyl groups or a substituted phenyl group. In a preferred embodiment of the invention, R represents a substituted phenyl group having at least one substituent selected from the group consisting of methyl ester, amino, amido, acyloxy and thio groups. In another preferred embodiment, R represents a straight or branched chain alkyl group having 1 to 6 carbon atoms, said alkyl group having a substituent selected from the group consisting of sulfhydryl and phenyl groups. Additionally, there are provided compound which are functional analogs of the compound of formula I compound.

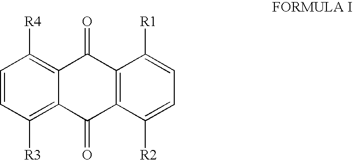

As aforementioned, therapeutic compositions of the invention are effective at dosages that substantially eliminate the adverse inflammatory or irritancy effects associated with the use of anthraquinone and related compounds. Accordingly, there is provided a therapeutic composition comprising a therapeutically effective amount of at least one compound of the invention and a pharmaceutically acceptable carrier. These compounds of the invention have anti-proliferative effects, antineoplastic effect, allergic, inflammatory conditions, tumor condition, stem cell application, tissue engineering and therapeutic compositions containing such compounds. Further additional representative and preferred aspects of the invention are described below according to the following detailed description of the invention.

### DESCRIPTION OF THE DRAWINGS

FIG. 1 shows structures of some prior art of anthraquinone derivatives.

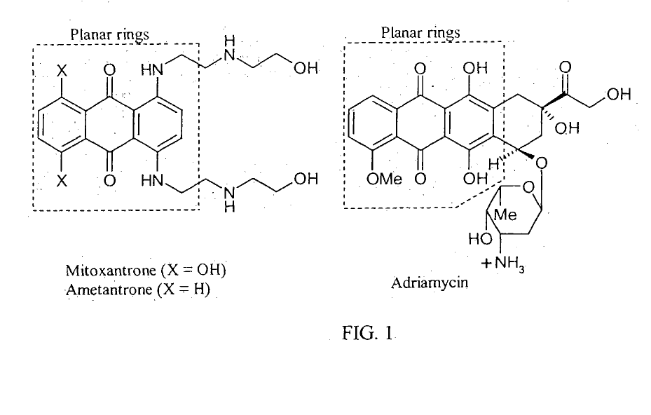

FIG. 2 is a schematic drawing of the general synthetic method used to prepare the novel 1,5-bisthioanthraquinone derivatives of the inventions. The synthesis reagents include: sodium methoxide/methanol; tetrahydrofuran.

FIG. 3 is a schematic drawing of the general synthetic method used to prepare the novel 1,5-bisacyloxy anthraquinone derivatives of the inventions. The synthesis reagents include: (a) pyridin; or (b) NaH, tetrahydrofuran.

FIG. 4 is a schematic drawing of the general synthetic method used to prepare the novel 1,5-bisaminoanthraquinone derivatives of the inventions. The synthesis reagents include: DMF, heated in the glass mini-reactor.

FIG. 5 is a schematic drawing of the general synthetic method used to prepare the novel 1,8-bisaminoanthraquinone derivatives of the inventions. The synthesis reagents include: DMF, heated in the glass mini-reactor.

FIG. 6 is a schematic drawing of the general synthetic method used to prepare the novel 1,4-bisamidoanthraquinone derivatives of the inventions.

FIG. 7 is a schematic drawing of the general synthetic method used to prepare the novel 1,5-bisamidoanthraquinone derivatives of the inventions.

FIG. 8 is an outline of the synthesis of the bis-substituted anthraquinone derivatives of the inventions.

### DETAILED DESCRIPTION OF THE INVENTION

As set forth above, the compounds of the invention are bis-substituted anthraquinone analogues. According to of the invention, there are provided bis-substituted anthraquinone derivatives according to Formula I, wherein R1, R2, R3, and R4 represent a straight or branched chain alkyl group having 1 to 6 carbon atoms, acyloxy-substituted, alkylthio-substituted, amido-substituted, and amino-substituted, phenyl or benzyl, wherein the alkyl group may be substituted with one or more groups Ra, Rb, Rc, Rd, and the phenyl or benzyl group may be substituted with one or two groups Ra, Rb, Rc, Rd. Ra, Rb, Rc, Rd are group selected from halogen, NH₂, NO₂, OH, CH₃—, CH₃CH₂—, CH₃CH₂CH₂— and CH₃CH₂CH₂CH₂—, CH₃CH₂CH₂CH₂CH₂—. Ra, Rb, Rc, Rd are group selected from a straight or branched chain alkyl group having 1 to 5 carbon atoms, halogen, NH₂, NO₂, OH, CH₃—, CH₃CH₂—, CH₃CH₂CH₂— and CH₃CH₂CH₂CH₂—, CH₃CH₂CH₂CH₂CH₂—.

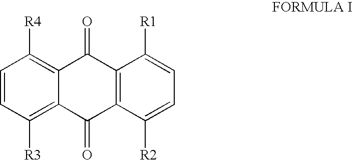

In preferred compounds according to the invention, R1, R2, R3, and R4 represent a straight or branched chain alkyl group having 1 to 6 carbon atoms which may be substituted with one or more groups Ra, Rb, Rc, Rd, selected from C₁, NH₂, OH, NO₂, CH₃O, cyclopentane, cyclohexane. In other preferred embodiments, R1, R2, R3, and R4 are phenyl or benzyl group having one or two substituents Ra, Rb, Rc, Rd, selected from a straight or branched chain alkyl group having 1 to 6 carbon atoms, halogen, NH₂, OH, NO₂, CH₃O. Suitable compounds of the invention described in Table 1-4.

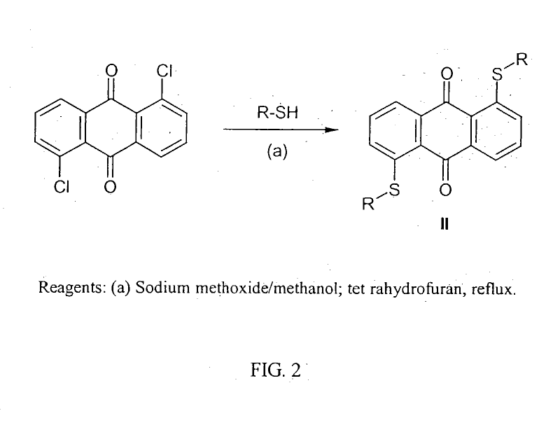

In the course of synthesis of 1,5-bisthioanthraquinons, it was found that the reaction undergoes a nucleophilic substitution at the 1 and 5 positions with the appropriate thiols in the presence of sodium methoxide and THF at room temperature or after reflux for 1-2 h to generate this structural class of anthraquinones. (FIG. 2)

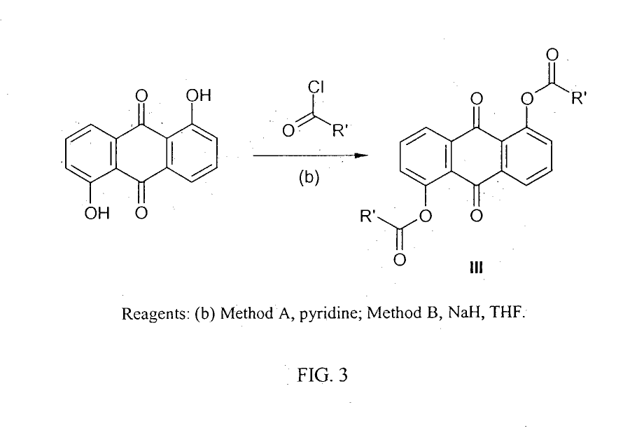

The synthesis of 1,5-bisacyloxy-anthraquinone derivatives shown in FIG. 3. The method of preparation of the 1,5-bisacyloxy anthraquinones was based on that of simple acylation involving 1,5-dihydroxyanthraquinone (anthrarufin) with an excess of the appropriate acyl chlorides in the presence of pyridine and dichloromethane at room temperature for 1 to 2 hours; or in the presence of NaH and THF at room temperature or reflux for 1 to 2 hours. Accordingly, acylation of the appropriately anthraquinones with the appropriate acyl chloride gave the bis-substituted anthraquinones in essentially quantitative yield.

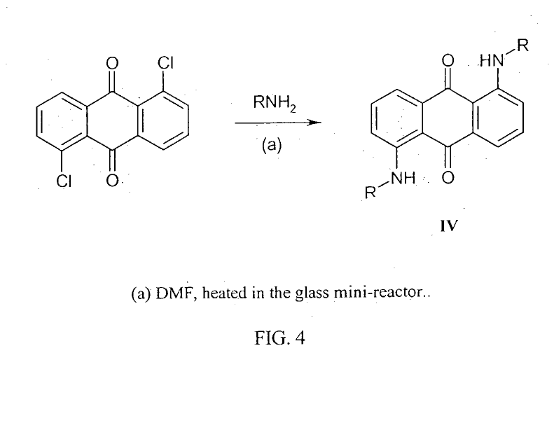

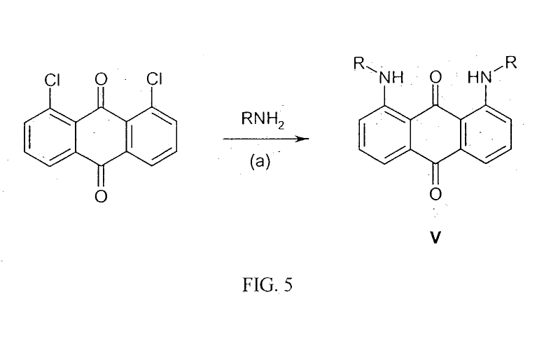

The 1,5-bisaminoanthraquinones and 1,8-bisaminoanthraquinones were synthesized by heating 1,5-dichloroanthraquinone or 1,8-dichloroanthraquinone with a large excess of various amines in the glass mini-rector. Treatment of start materials with substituted amines in DMF resulted in the replacement of two halogen atom by the amino group to yield mainly symmetrical bisaminosubstituted anthraquinones. This method involves nucleophilic displacement of start materials by various amine derivatives to form bis-substituted anthraquinones. This procedure (FIG. 4 and FIG. 5) was satisfactory for the preparation of higher homologues. The overall yields and purities of the bisaminosubstituted products were generally better after recrystallization.

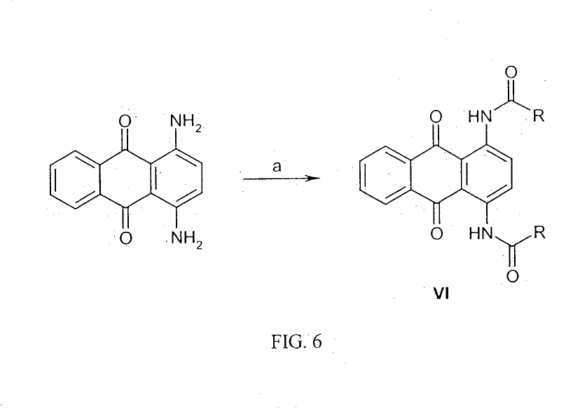

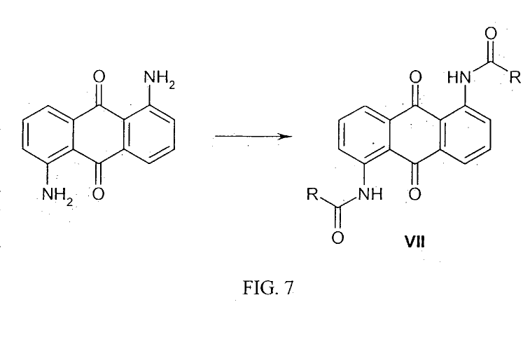

In the course of synthesis of 1,4- and 1,5-bisamidoanthraquinons, it was found that the reaction undergoes a nucleophilic substitution at the 1,4 and 1,5 positions with the appropriate acyl chlorides in the presence of pyridine and N,N-diethylacetamide at room temperature for 24 h or after reflux for 1-2 h to generate this structural class of anthraquinones. (FIG. 6 and FIG. 7)

For the pharmaceutical compositions according to the invention, salts of anthraquinone compounds are in particular salts with the pharmaceutically acceptable base. Excipients such as magnesium stearate, corn starch, starch, lactose, sodium hydroxymethylcellulose, ethanol, glycerol etc. may be added in the preparation of pharmaceutical compositions containing bissubstituted anthraquinone derivatives of the present invention. The pharmaceutical compositions of the invention may be in an injectable form or formulated into tablet, pill or other solid preparation forms. The pH value for injectable forms may be adjusted with phosphate buffer. Generally, dosage used for injectable forms is 25-100 mg. For solid preparations, an effective dosage is 3-500 mg, administered 2 to 3 times a day.

### Clinical Indications Subject to Treatment

The following conditions are selected for description herein as being representative of inflammatory, allergic, antioxidant; stem cell application, tissue engineering, delay age-associate tissue degeneration, reverse organ failure in chronic high-turnover disease, or neoplastic conditions that are suitable for treatment according to the practice of the invention. Each of these conditions involves intimation hyperproliferation and/or generation of free radicals and active oxygen species.

### Neoplastic Conditions

The therapeutic compositions of the invention may be used in the treatment of a wide variety of cancers such as carcinomas, sarcomas, melanomas, hepatoma and lymphomas, which may affect a wide variety of organs, including, for example, the lung, mammary tissue, prostate gland, small or large intestine, liver, heart, skin, pancreas and brain. The therapeutic compositions may be administered by injection (intravenously, intralesionally, peritoneally, subcutaneously), or by topical application and the like as would be suggected according to the routine practice of the art.

### Telomerase Activity

The telomerase activating compounds should be valuable in the fields of stem cell and tissue engineering research in expending target cells. They may also be applied in treating age-associated tissue degeneration or reverse organ failure in chronic high-turnover diseases. This unique property should definitely be noted in future drug design. Because telomerase expression is a hallmark of cancer, the effect of anthraquinones on telomerase expression was determined. The telomerase activity is regulated mostly at the transcriptional level for its catalytic subunit, hTERT, and partly at the post-translational level. Since the expression of human telomerase catalytic component is the key regulator in telomerase activity, we analyzed the expression of telomerase by monitoring the expression of hTERT as the criteria. Thus, inhibition or activation of the reverse transcriptase telomerase can profoundly affect the proliferative capacity of normal cells and cancers. In addition to anticancer functions, our finding raises the possibility that these compounds might also have a role in cell immortilization. The application of these anthraquinone derivatives in stem cell research and tissue engineering is also discussed.

### Psoriasis and Contact Dermatitis

Psoriasis is a widespread, chronic, inflammatory and scaling skin disease. Contact dermatitis, in contrast, is a short term allergic condition characterized by scaling skin. Both psoriasis and contact dermatitis are characterized by increased epidermal cell proliferation at the affected site or sites, i.e. lesions.

Arthritic Disease

Rheumatoid arthritis is chronic inflammatory disease, primarily of the joints, that may result in permanent loss of joint function. Irreversible loss of joint function is attributed to severe degradation of collagen, bone, ligament and tendon. Associated chronic intimation results, in part, from immune response at the affected joint, although the exact nature of the triggering antigens is unknown. The immune response may be autoimmune in origin. Briefly, there is a progressive loss of cartilage (a connective tissue) caused by invading cells. Both collagen and proteoglycan components of the cartilage are degraded by enzyme released at the affected site.

### Therapeutic Compositions and Administration Thereof

The amount of bis-substituted anthraquinones (or salt thereof) administered for the prevention or inhibition of an inflammatory or allergic condition, for antiproliferative, telomerase activity, stem cell, applied in treating age-associated tissue degeneration or reverse organ failure in chronic high-turnover diseases or antineoplastic effect, can be determined readily for any particular patient according to recognized procedures. Additional information useful in the selection of therapeutic compositions is provided as follows. For use in the treatment of inflammatory or degenerative conditions, as those term are recognized in the art, the therapeutic compositions may be administered, for example, by injection at the affected site, by aerosol inhalation (as in the case of emphysema or pneumonia), or by topical application or transdermal absorption as would also be suggested according to the routine practice of the art.

As described above, the bis-substituted anthraquinones (or salt thereof) may be incorporated into a pharmaceutically acceptable carrier or carrier for application (directly or indirectly) to the affected area. The nature of the carrier may vary widely and depend on the intended location of application and other factors well known in the art. Such carrier of anthraquinone derivatives are well known in the art.

### Preparation of the Compounds of the Invention

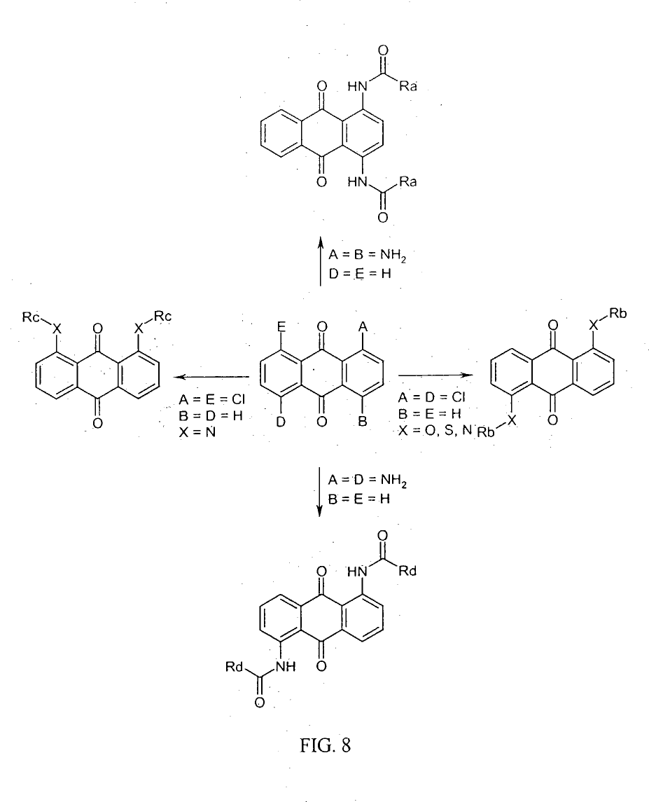

FIG. 8 is an outline of a synthesis of the bis-substituted anthraquinone compounds (Formula I) according to the invention. The synthesis of 1,5-bis-thio-anthraquinone derivatives shown in FIG. 2 and FIG. 8 were accomplished using procedures somewhat modified from those described elsewhere. The reaction undergoes a nucleophilic substitution at the 1 and 5 positions with the appropriate thiols in the presence of sodium methoxide and THF at room temperature or after reflux for 1 to 2 h to generate this structural class of anthraquinones. The mechanism for the reaction may be rationalized assuming that thiols are ionized by sodium methoxide as nucleophiles undergo nucleophilic substitution.

As shown in FIG. 3 and FIG. 8, the preparation of the symmetrical bis-substituted anthraquinones was based on that of simple acylation involving 1,5-dihydroxyanthraquinone (anthrarufin) with an excess of the appropriate acyl chlorides in the presence of pyridine and dichloromethane at room temperature for 1 to 2 hours; or in the presence of NaH and THF at room temperature or reflux for 1 to 2 hours. Accordingly, acylation of the appropriately anthraquinones with the appropriate acyl chloride gave the bis-substituted anthraquinones in essentially quantitative yield. Specific methods for the preparation of several compounds or different position substitution according to the present invention are described below in Example 1. The structure of each of the synthesized compounds is confirmed by 1H-NMR spectrometry, mass spectrometry, UV and IR as shown in Example 2. Procedures adapted from the descriptions and the following non-limiting examples will allow one skilled in the art to prepared similar compounds of the invention.

### EXAMPLES

The following non-limiting examples are representative of the practice of the invention.

### Example 1

### Methods of Synthesis

The novel bis-substituted anthraquinone compounds described in Table 1-4 were produced as follow.

General Procedure for the Preparation of 1,5-bis-thio-anthraquinones (II). To a solution of 1,5-dichloroanthraquinone (1.0 g, 3.6 mmol) in dry THF (100 ml) a solution of an appropriate thiols (28.8 mmol) in sodium methoxide (1.56 g, 28.8 mmol) and dry methanol (30 ml) under N₂ was added dropwise. The reaction mixture was refluxed for 1 h. Water (250 ml) was added, and then the mixture was extracted with dichloromethane. The combined organic extracts were washed with water, dried (MgSO₄), and concentrated. The resulting precipitate was collected by filtration, washed with water and further purified by chromatography and crystallization.

General procedure for the preparation of 1,5-bisacyloxy anthraquinones (III). Method A: To a solution of anthrarufin (4.25 mmol) and pyridine (20 ml) in dry CH₂Cl₂ (150 ml) was added dropwise a solution of an appropriate acyl chlorides (10 mmol) in dry CH₂Cl₂ (10 ml) at 0° C. under N₂. The reaction mixture was stirred or refluxed for 1-2 hours. Water (250 ml) was added and then extracted with dichloromethane. The combined organic extracts were washed with water and dried (MgSO₄), and concentrated. The resulting precipitate was collected by filtration, washed with water and further purified by crystallization and chromatography.

Method B: To a solution of anthrarufin (4.25 mmol) in dry THF (20 ml) and NaH (12.75 mmol) was added dropwise a solution of an appropriate acyl chlorides (3 mmol) in dry THF (10 ml) at 0° C. under N₂. The reaction mixture was stirred or refluxed for 1-2 hours. Water (250 ml) was added and then extracted with dichloromethane. The combined organic extracts were washed with water and dried (MgSO₄), and concentrated. The resulting precipitate was collected by filtration, washed with water and further purified by crystallization and chromatography.

General Procedure for the Preparation of 1,5-diaminoanthraquinones (IV). A mixture of 1,5-dichloroanthraquinone (1.0 g, 3.6 mmol) and DMF (20 ml) containing an appropriate amine (8.0 mmol) was heated in a glass mini-reactor for 30 minutes. The reaction mixture was treated with crushed ice. The resulting precipitate was collected by filtration, washed well with water. Recrystallization from ethylacetate and n-hexane afforded the final product as red needles.

General Procedure for the Preparation of 1,8-diaminoanthraquinones. (V). A mixture of 1,8-dichloroanthraquinone (1.0 g, 3.6 mmol) and DMF (20 ml) containing an appropriate amine (8.0 mmol) was heated in a glass mini-reactor for 30 minutes. The reaction mixture was treated with crushed ice. The resulting precipitate was collected by filtration, washed well with water. Recrystallization from ethylacetate and n-hexane afforded the final product as dark red needles.

General Procedure for the Preparation of 1,4-diamidoanthraquinones (VI). A solution of 1.0 g (4 mol) of 1,4-diaminoanthraquinone in 70 mL of N,N-diethylacetamide was cooled to 0° C., and then 0.5 mL (4 mol) of pyridine and 1.00 mL (12 mol) of chloroacetylchloride was slowly with vigorous stirring. The reaction mixture was further stirred for 24 h at room temperature. The product was precipitated by treatment of diethyl ether and then the filtrate washed carefully with diethyl ether. The crude product was recrystallized from ethyl acetate-n-hexane to afford pure compounds.

1,4-bis-(2-chloroacetamidoamido)-9,10-anthraquinone 0.39 g (1 mmol) was suspended in 70 ml of EtOH with stirring. The mixture was warmed to 70° C. and 1.50 ml (20 mmol) of diethylamine (added 20 ml of EtOH) was added dropwise over a I-h period. After 18 h, the solvent was removed under reduced pressure to yield a brown/black solid. After crystallization from ethyl acetate/n-hexane which were suspended in ethanol, and 2 equiv (mM) of aqueous hydrogen chloride was added. The solvent was stirred for 30 min and filtered. The resulting brown solution was dried to give brown powder.

General Procedure for the Preparation of 1,5-diamidoanthraquinones (VII). A solution of 0.24 g (1 mmol) of 1,5-diaminoanthraquinone in 25 mL of N,N-dimethylformamide was cooled to 0° C., and then 0.5 mL (4 mol) of pyridine and 1.00 mL (3 mmol) of chloroacetylchloride was slowly with vigorous stirring. The reaction mixture was further stirred for 24 h at room temperature. The product was precipitated by treatment of diethyl ether and then the filtrate washed carefully with diethyl ether. The crude product was recrystallized form ethyl acetate/n-hexane to afford desired compounds.

### Example 2

### Structural Confirmation

Melting points were determined with a Büchi B-545 melting point apparatus and are uncorrected. All reactions were monitored by TLC (silica gel 60 F₂₅₄), flash-column chromatography: silica gel (E. Merck, 70-230 mesh) with CH₂Cl₂ as the eluent. ¹H-NMR: Varian GEMINI-300 (300 MHz) and Brucker AM-500 (500 MHz); δ values are in ppm relative to TMS as an internal standard. Fourier-transform IR spectra (KBr): Perkin-Elmer 983G spectrometer. The UV spectra were recorded on a Shimadzu UV-160A. Mass spectra (EI, 70 eV, unless otherwise stated): Finnigan MAT TSQ46 and Finnigan MAT TSQ-700 (Universität Regensburg, Germany). Typical experiments illustrating the general procedures for the preparation of the anthraquinones are described below.

1,5-Bis(ethylthio)-anthraquinone (IIa). The compound was synthesized as Example 1 and analyzed: 66% yield. m.p. 235-236° C. (THF). ¹H-NMR (CDCl₃) δ: 1.45 (6H, t, J=7.4 Hz), 3.01 (4H, q, J=7.4 Hz), 7.60 (2H, d, J=8.0 Hz), 7.66 (2H, t, J=7.8 Hz), 8.11 (2H, t, J=7.6, 0.9 Hz). ¹³C-NMR (CDCl₃) δ: 12.77, 25.96, 123.47, 127.89, 129.26, 133.14, 136.09, 145.03, 183.33. IR (KBr) cm⁻¹: 1651, 1202. UV λₘₐₓ (CHCl₃) nm (log ε): 503 (2.41). MS m/z: 328 (M⁺), 299, 267, 239, 139. Anal. Calcd. for C₁₈H₁₆O₂S₂: C, 65.82; H, 4.91.

Found: C, 65.65; H, 4.88.

1,5-Bis(hydroxyethylthio)-anthraquinone (IIb). The compound was synthesized as Example 1 and analyzed: 45% yield. m.p. 261-7262° C. (DMSO). ¹H-NMR (CDCl₃) δ: 3.12 (4H, t, J=6.5 Hz), 3.70 (4H, q, J=6.2 Hz), 5.04 (2H, t, J=5.5 Hz), 7.78 (2H, d, J=7.6 Hz), 7.82-7.80 (2H, m), 7.94 (2H, dd, J=6.8, 1.5 Hz). ¹³C-NMR (CDCl₃) δ: 34;04, 59.06, 122.84, 127.42, 129.88, 133.57, 135.55, 144.07, 182.35. IR (KBr) cm⁻¹: 1638, 1204. UV λₘₐₓ (CHCl₃) nm (log ε): 5.13 (2.48). MS m/z: 360 (M⁺), 324. Anal. Calcd. for C₁₈H₁₆O₄S2: C, 59.98; H, 4.47. Found: C, 59.81; H, 4.38.

1,5-Bis(propylthio)-anthraquinone (IIc). The compound was synthesized as Example 1 and analyzed: 69% yield. m.p. 232-233° C. (THF). ¹H-NMR (CDCl₃) δ: 1.13 (6H, t, J=7.4 Hz), 1.83 (4H, m), 2.96 (4H, t, J=7.4 Hz), 7.60 (2H, d, J=7.9 Hz), 7.65 (2H, t, J=7.8 Hz), 8.11 (2H, d, J=6.9 Hz). ¹³C-NMR (CDCl₃) δ: 13.96, 21.33, 34.04, 123.46, 127.99, 129.32, 13312, 136.14, 145.20, 183.35. IR (KBr) cm⁻¹: 1649, 1199. UV λₘₐₓ (CHCl₃) nm (log ε): 485 (2.25). MS m/z: 356 (M⁺), 313, 271, 239, 139. Anal. Calcd. for C₂₀H₂₀O₂S₂: C, 67.38; H, 5.65. Found: C, 67.55; H, 5.78.

1,5-Bis(dihydroxypropylthio)-anthraquinone (IId). The compound was synthesized as Example 1 and analyzed: 45% yield. m.p. 238-239° C. (DMSO). ¹H-NMR (CDCl₃) δ: 2.93 (2H, t, J₁=10.1 Hz), 3.20 (2H, dd, J=12.7, 4.2 Hz), 3.41-3.50 (4H, m), 3.73 (2H, m), 4.79 (2H, t), 5.12 (2H, d, J=5.3 Hz), 7.79 (2H, t, J=7.7 Hz), 7.82 (2H, d, J=7.4 Hz), 7.94 (2H, t, J=7.3, 0.8 Hz). ¹³C-NMR (CDCl₃) δ: 35.53, 65.05, 69.91, 122.75, 127.39, 130.02, 133.56, 135.55, 144.71, 182.41. IR (KBr) cm⁻¹: 1647, 1202. UV λₘₐₓ (CHCl₃) nm (log ε): 507 (2.48). MS m/z: 420 (M⁺), 348. Anal. Calcd. for C₂₀H₂₀O₆S₂: C, 57.12; H, 4.79. Found: C, 57.35; H, 4.98.

1,5-Bis(hydroxyhexylthio)-anthraquinone (11e). The compound was synthesized as Example 1 and analyzed: 79% yield. m.p. 195-196° C. (DMSO). ¹H-NMR (CDCl₃) δ: 1.37 (41H, q, J=6.9 Hz), 1.46 (4H, q, J=6.9 Hz), 1.49 (4H, q, J=7.5 Hz), 1.70 (4H, q, J=7.3 Hz), 3.00 (4H, t, J=7.2 Hz), 3.41 (4H, q, J=5.9 Hz), 4.10 (2H, t, J=5.1 Hz), 7.77-7.80 (4H, m), 7.95 (21H, d, J=6.5 Hz). ¹³C-NMR(CDCl₃) δ: 24.79, 27.31, 28.10, 30.77, 32.04, 60.41, 122.41, 127.18, 129.64, 133.13, 135.34, 144.10, 181.98. IR (KBr) cm⁻¹: 1643, 1259. UV λₘₐₓ (DMSO) nm (log ε): 564 (0.32). MS m/z: 472 (M⁺), 474. Anal. Calcd. for C₂₆H₃₂O₄S₂: C, 66.06; H, 6.82. Found: C, 66.35; H, 6.98.

1,5-Bis(o-aminophenylthio)-Anthraquinone (IIf). The compound was synthesized as Example 1 and analyzed: 55% yield. m.p. 283-284° C. (DMSO). ¹H-NMR (CDCl₃) o: 5.37 (4H, s), 6.66 (2H, t, J=7.5 Hz), 6.85 (2H, d, J-=8.1 Hz), 7.01 (2H, d, J=8.2 Hz), 7.25 (2H, t, J=7.6, 0.9 Hz), 7.34 (2H, d, J=7.5 Hz), 7.66 (2H, t, J=7.9 Hz), 8.00 (2H, d, J=7.4 Hz). ¹³C-NMR(CDCl₃) δ: 111.35, 115.06, 117.03, 123.67, 127.77, 130.43, 131.77, 133.41, 135.51, 137.08, 143.20, 150.66, 182.65. IR (KBr) cm⁻¹: 1651, 1256. UV λₘₐₓ (DMSO) nm (log ε): 508 (2.36). MS m/z: 454 (M⁺), 361. Anal. Calcd. for C₂₆H₁₈N₂O₂S₂: C, 68.69; H, 3.99. Found: C, 68.55; H, 3.78.

1,5-Bis(m-aminophenylthio)-Anthraquinone (IIg). The compound was synthesized as Example 1 and analyzed: 65% yield. m.p. 292-293° C. (DMSO). ¹H-NMR (CDCl₃) o: 5.40 (4H, s), 6.71-6.73 (4H, m), 6.80 (2H, s), 7.16 (2H, d, J=8.3 Hz), 7.19 (2H, t, J=7.8 Hz), 7.67 (2H, t, J=7.9 Hz), 7.97 (2H, d, J=7.5 Hz). ¹³C-NMR (CDCl₃) δ: 115.41, 120.04, 122.24, 123.57, 126.59, 130.74, 130.94, 131.01, 133.49, 135.10, 145.53, 150.44, 182.42. IR (Ker) cm⁻¹: 1653, 1202. UV λₘₐₓ (DMSO) nm (log ε): 535 (2.48). MS m/z: 454 (M⁺), 125. Anal. Calcd. for C₂₆H₁₈N₂O₂S₂: C, 68.69; H, 3.99. Found: C, 68.49; H, 3.69.

1,5-Bis(p-aminophenylthio)-Anthraquinone (IIh). The compound was synthesized as Example 1 and analyzed: 66%-yield. m.p. 364-365° C. (DMSO). ¹H-NMR (CDCl₃) δ: 5.64 (4H, s), 6.70 (4H, t, J=8.3 Hz), 7.07 (2H, d, J=8.3 Hz), 7.20 (4H, d, J=8.3 Hz), 7.63 (2H, t, J=7.9 Hz), 7.94 (2H, d, J=7.5 Hz). ¹³C-NMR (CDCl₃) δ: 114.01, 115.19, 123.28, 126.46, 130.57, 133.29, 135.19, 137.02, 147.69, 150.64, 182.41. IR (KBr) cm⁻¹: 1649, 1283. UV λₘₐₓ (DMSO) nm (log ε): 557 (2.48). MS m/z: 454 (M⁺), 124. Anal. Calcd. for C₂₆H₁₈N₂O₂S₂: C, 68.69; H, 3.99. Found: C, 68.49; H, 3.68.

1,5-Bis(benzylthio)-anthraquinone (IIi). The compound was synthesized as Example 1 and analyzed: 78% yield. m.p. 281-282° C. (THF). ¹H-NMR (CDCl₃) δ: 4.23 (4H, s), 7.27 (2H, t, J=7.3 Hz), 7.33 (4H, d, J=7.4 Hz), 7.45 (4H, d, J=7.4 Hz), 7.62 (2H, d, J=8.0 Hz), 7.66 (2H, t, J=7.4 Hz), 8.10 (2H, d, J=7.1 Hz). ¹³C-NMR (CDCl₃) o: 37.35, 123.76, 127.58, 127.91, 128.78, 129.10, 129.59, 133.32, 135.41, 135.86, 144.92, 183.31. IR (KBr) cm⁻¹: 1653, 1261. UV λₘₐₓ (CHCl₃) nm (log ε): 476 (1.50). MS m/z: 452 (M⁺), 361, 270, 91. Anal. Calcd. for C₂₈H₂₀O₂S₂: C, 74.30; H, 4.55. Found: C, 74.55; H, 4.78.

1,5-Bis(p-methoxybenzylthio)-Anthraquinone (IIj). The compound was synthesized as Example 1 and analyzed: ⁶²% yield. m.p. 297-299° C. (THF). HR-FAB-MS m/z: 512.6472 (calcd. for C₃₀H₂₄O₄S₂: 512.6492).

1,5-Bis(phenylethylthio)-anthraquinone (IIk). The compound was synthesized as Example 1 and analyzed: 69% yield. m.p. 209-210° C. (THF). ¹H-NMR (CDCl₃) o: 3.08 (4H, t, J 8.0 Hz), 3.25 (4H, t, J=8.1 Hz), 7.28 (2H, t, J=7.0 Hz), 7.30 (2H, t, J=8.3 Hz), 7.32 (2H, d, J=7.4 Hz), 7.62 (2H, d, J=7.4 Hz), 7.66 (2H, t, J=7.7 Hz), 8.12 (2H, d, J=6.2 Hz).

¹³C-NMR(CDCl₃) δ: 33.64, 34.28, 123.65, 126.68, 128.02, 128.43, 128.69, 129.31, 133.22, 136.07, 140.04, 144.64, 183.29. IR (KBr) cm⁻¹: 1653, 1204. UV λₘₐₓ (CHCl₃) nm (log ε): 512 (0.60). MS m/z: 480 (M⁺), 285. Anal. Calcd. for C₃₀H₂₄O₂S₂: C, 74.96; H, 5.03. Found: C, 74.75; H, 4.91.

1,5-Bis(propionyloxy)-anthraquinone (IIIa). The title compound was obtained from anthrarufin and acetyl chloride according to Method A. Recrystallization from ethanol gave yellow needles; 55% yield; m.p. 230-231° C.; ¹H-NMR (CDCl₃) δ 1.34 (t, J=7.5 Hz, 6H), 2.80 (q, J=7.5 Hz, 4H), 7.37 (d, J=8.1 Hz, 2H), 7.75 (t, J=8.0 Hz, 2H), 8.16 (d, J=7.7 Hz, 2H); FTIR (KBr): 1759, 1674 cm⁻¹; UV λₘₐₓ (CHCl₃) nm (log ε): 318 (2.48); MS m/z 352 (4, M⁺), 296 (23), 240 (100).

1,5-Bis(butyryloxy)-anthraquinone (IIIb). The title compound was obtained from anthrarufin and butyryl chloride according to Method A. Recrystallization from ethanol gave yellow needles; 59% yield; m.p. 211-213° C.; ¹H-NMR (CDCl₃) δ 1.1 (t, J=7.4 Hz, 6H), 1.83-1.90 (m; 4H), 2.75 (t, J=7.5 Hz, 4H), 7.36 (d, J=8.0 Hz; 2H), 7.74 (t, J=7.9 Hz, 2H), 8.16 (d, J=7.8 Hz, 2H); ¹³C-NMR (CDCl₃) δ 181.14, 172.03, 150.08, 135.93, 134.88, 129.65, 125.63, 124.45, 36.15, 18.04, 13.75; UV λₘₐₓ (CHCl₃) nm (log ε): 319 (2.51); FTIR (KBr) 1757, 1676 cm⁻¹; MS m/z 380 (3, M⁺), 310 (22), 240 (100).

1,5-Bis(hexanoyloxy)-anthraquinone (IIIc). The title compound was obtained from anthrarufin and hexanoyl chloride according to Method A. Recrystallization from ethanol gave yellow needles; 74% yield; m.p. 183-184° C.; ¹H-NMR (CDCl₃) δ 0.95 (t, J=7.1 Hz, 6H), 1.38-1.49 (m, 8H), 1.84 (q, J=7.4 Hz, 4H), 2.76 (t, J=7.7 Hz, 4H), 7.40 (dd, J 7.8, 1.0 Hz, 2H), 7.74 (t, J=8.1, 7.8 Hz, 211), 8.16 (t-like, J=7.7, 2.3 Hz, 2H); ³C-NMR (CDCl₃) δ 181.14, 172.22, 150.10, 135.93, 134.87, 129.64, 125.63, 124.45, 34.26, 31.35, 24.18, 22.39, 13.97; UV λₘₐₓ (CHCl₃) nm (log ε) 318 (2.44); FTIR(KBr) 1755, 1676 cm⁻¹; MS m/z 436 (4, M⁺), 338 (24), 240 (100); Anal. C₂₆H₂₈O₆ (C, H).

1,5-Bis(pivaloyloxy)-anthraquinone (IIId). The title compound was obtained from anthrarufin and pivaloyl chloride according to Method B. Recrystallization from ethanol gave yellow needles; 25% yield; m.p. 166-167° C.; ¹H-NMR (CDCl₃) δ 1.47 (s, 18H), 7.31 (d, J=8.1 Hz, 2H), 7.72 (d, J=8.0 Hz, 2H), 8.16 (d, J=7.5 Hz, 2H); ¹³C-NMR (CDCl₃) δ 181.00, 176.66, 150.40, 135.98, 134.62, 129.38, 125.59, 124.78, 39.21, 27.23; UV λₘₐₓ (EtOH) nm (log ε) 363 (1.40); FTIR (KBr) 1751, 1670 cm⁻¹; MS m/z 408 (3, M⁺), 324 (23), 240 (100).

1,5-Bis(benzoyloxy)-anthraquinone (IIIe). The title compound was obtained from anthrarufin and benzoyl chloride according to Method A. Recrystallization from ethanol gave yellow needles; 76% yield; m.p. 336-338° C. (lit.^{[13]} mp 342° C.); ¹H-NMR (CDCl₃) δ 7.50 (d, J=7.9 Hz, 2H), 7.56 (t, J=7.7 Hz, 4H), 7.68 (t, J=7.3 Hz, 2H), 7.77 (t, J=7.9 Hz, 2H), 8.17 (d, J=7.7 Hz, 2H), 8.29 (d, J=7.7 Hz, 4H); UV λₘₐₓ (CHCl₃) nm (log ε) 340 (0.69); FTIR (KBr) 1734, 1672 cm⁻¹; MS m/z 448 (4, M⁺), 105 (100); Anal. C₂₈H₁₆O₆ (C, H).

1,5-Bis(2-chlorobenzoyl)-anthraquinone (IIIf). The title compound was obtained from anthrarufin and o-chlorobenzoyl chloride according to Method B. Recrystallization from THF gave yellow needles; 39% yield; m.p. 254-255° C.; ¹H-NMR (CDCl₃) δ 7.47-7.55 (m, 8H), 7.80 (t, J=7.9 Hz, 2H), 8.22 (d, J=7.8 Hz, 2H), 8.39 (d, J=7.7 Hz, 2H); UV λₘₐₓ (CHCl₃) nm (log s) 334 (2.20); FTIR (KBr) 1747, 1672 cm⁻¹; MS m/z 516 (2, M⁺), 139 (100).

1,5-Bis(3-chlorobenzoyl)-anthraquinone (IIIg). The title compound was obtained from anthrarufin and m-chlorobenzoyl chloride according to Method B. Recrystallization from THF gave yellow needles; 49% yield; m.p. 301-302° C.; ¹H-NMR (CDCl₃) δ 7.50-7.52 (m, 4H), 7.65 (d, J=7.4 Hz, 2H), 7.79 (t, J=7.9 Hz, 2H), 8.16-8.19 (m, 4H), 8.26 (s, 2H); UV λₘₐₓ (CHCl₃) nm (log ε) 351 (0.33); FTIR (KBr) 1744, 1674 cm¹; MS m/z 516 (5, M⁺), 141 (35), 139 (100); Anal. C₂₈H₁₄Cl₂O₆ (C, H).

1,5-Bis(4-chlorobenzoyl)-anthraquinone (IIIh). The title compound was obtained from anthrarufin and p-chlorobenzoyl chloride according to Method B. Recrystallization from THF gave yellow needles; 69% yield; m.p. 327-328° C.; ¹H-NMR (CDCl₃) δ 7.50 (d, J=7.9 Hz, 2H), 7.54 (d, J=8.4 Hz, 4H), 7.78 (t, J=7.9 Hz, 2H), 8.16 (d, J=7.9 Hz, 2H), 8.22 (d, J=8.4 Hz, 4H); UV λₘₐₓ (CHCl₃) nm (log ε) 351 (1.77); FTIR (KBr) 1736, 1676 cm⁻¹; MS m/z 516 (2, M⁺), 139 (100); Anal. C₂₈H₁₄Cl₂O₆ (C, H).

1,5-Bis(2,4-dichlorobenzoyl)-anthraquinone (IIIi). The title compound was obtained from anthrarufin and o,p-dichlorobenzoyl chloride according to Method B. Recrystallization from THF gave yellow needles; 38% yield; m.p. 310-312° C.; IR (KBr) 1740, 1668 cm⁻¹; MS m/z 586 (4, M⁺), 421 (25), 240 (56), 173 (100); HRMS m/z: Calcd. for C₂₈H₁₂Cl₄O₆: 514.1464. Found: 514.1478.

1,5-Bis(2-toluoyloxy)-anthraquinone (IIIj). The title compound was obtained from anthrarufin and o-toluoyl chloride according to Method A. Recrystallization from THF gave yellow needles; 68% yield; m.p. 262-263° C.; ¹H-NMR (CDCl₃) δ 2.68 (s, 6H), 7.35 (d, J=7.6 Hz, 2H), 7.39 (t, J=7.6 Hz, 2H), 7.49-7.53 (m, 4H), 7.78 (t, J=7.9 Hz, 2H), 8.19 (dd, J=8.1, 0.1 Hz, 2H), 8.35 (t, J=7.7, 0.7 Hz, 2H); ³C-NMR (CDCl₃) δ 181.15, 165.55, 150.21, 141.57, 136.01, 134.93, 132.86, 131.91, 131.69, 129.89, 128.39, 126.03, 125.84, 124.71, 21.76; UV λₘₐₓ (CHCl₃) nm (log ε) 314 (1.56); FTIR (KBr) 1736, 1674 cm¹; MS m/z 476 (2, M⁺), 119 (100). Anal. C₃₀H₂₀O₆ (C, H).

1,5-Bis(3-toluoyloxy)-anthraquinone (IIIk). The title compound was obtained from anthrarufin and m-toluoyl chloride according to Method B. Recrystallization from THF gave yellow needles; 28% yield; m.p. 269-270° C.; ¹H-NMR (CDCl₃) δ 2.47 (s, 6H), 7.44 (t, J=7.6 Hz, 2H), 7.48-7.50 (m, 4H), 7.76 (t, J 8.0 Hz, 2H), 8.09 (d, J=6.7 Hz, 4H), 8.17 (d, J=7.7 Hz, 2H); ¹³C-NMR (CDCl₃) δ 181.05, 165.28, 150.25, 138.52, 135.94, 134.91, 134.56, 130.96, 129.78, 129.34, 128.60, 127.67, 125.94, 124.60, 21.36; UV λₘₐₓ (CHCl₃) nm (log ε) 310 (1.84); FTIR (KBr) 1732, 1674 cm⁻¹; MS m/z 476 (4, M+, 119 (100); Anal. C₃₀H₂₀O₆: (C, H).

1,5-Bis(4-toluoyloxy)-anthraquinone (IIIl). The title compound was obtained from anthrarufin and p-toluoyl chloride according to Method B. Recrystallization from THF gave yellow needles; 39% yield; m.p. 331-332° C.; ¹H-NMR (CDCl₃) δ 2.47 (s, 6H), 7.36 (d, J=8.0 Hz, 4H), 7.49 (dd, J=7.8, 1.0 Hz, 2H), 7.75 (t, J=7.9 Hz, 2H), 8.17 (d, J=7.8 Hz, 4H); UV λₘₐₓ (CHCl₃) nm (log ε) 318 (0.90); FTIR (KBr) 1736, 1672 cm⁻¹; MS m/z 476 (5, M⁺), 119 (100); Anal. C₃₀H₂₀O₆ (C, H).

1,5-Bis(phenylacetyloxy)-anthraquinone (IIIm). The title compound was obtained from anthrarufin and phenylacetyl chloride according to Method A. Recrystallization from THF gave yellow needles; 35% yield; m.p. 202-203° C.; ¹H-NMR (CDCl₃) δ 4.10 (s, 4H), 7.30 (t, J=7.4 Hz, 2H), 7.33 (dd, J 8.0, 0.8 Hz, 2H), 7.37 (t, J=7.6 Hz, 4H), 7.46 (d, J=7.4 Hz, 4H), 7.74 (t, J=8.0 Hz, 2H), 8.18 (t, J 7.8, 0.8 Hz, 2H);

¹³C-NMR(CDCl₃)δ 181.06, 170.12, 149.99, 135.87, 134.95, 133.33, 129.76, 129.57, 128.63, 127.31, 125.83, 124.28, 41.13; UV λₘₐₓ (CHCl₃) nm (log δ) 318 (2.20); FTIR (KBr) 1763, 1670 cm⁻¹; MS m/z 358 (5, M⁺), 240 (94), 118 (100); Anal. C₃₀H₂₀O₆ (C, H).

1,5-Bis(phenylpropionyloxy)-anthraquinone (IIIn). The title compound was obtained from anthrarufin and phenylpropionyl chloride according to Method A. Recrystallization from THF gave yellow needles; 62% yield; m.p. 219-220° C.; ¹H-NMR (CDCl₃) δ 3.10 (s, 4H), 3.17 (t, J 7.9, 1.4 Hz, 4H), 7.22-7.35 (m, 12H), 7.74 (t, J=7.9 Hz, 2H), 8.16 (dd, J 7.7, 0.8 Hz, 2H); ¹³C-NMR (CDCl₃) δ 181.08, 171.35, 149.97, 140.39, 135.88, 134.96 , 129.62, 128.57, 128.47, 126.36, 125.75, 124.33, 35.86, 30.58; UV λₘₐₓ (CHCl₃) nm (log ε) 318 (1.10); FTIR (KBr) 1761, 1676 cm⁻¹; MS m/z 504 (5, M), 372 (10), 240 (100).

1,5-Bis(ethylamino)anthraquinone (IVa). The compound was synthesized as Example 1 and analyzed: 80% yield. mp 193-195° C. (EA/n-hexane). ¹H-NMR (CDCl₃) δ: 1.41-147 (6H, m), 3.39-3.47 (4H, q), 7.00 (2H, d, J=7.5 Hz), 7.53-7.58 (2H, m), 8.32 (2H, d, J=3.0 Hz), 9.65 (1H, br). ³C-NMR(CDCl₃) δ: 14.4, 37.4, 115.7, 116.2, 126.2, 133.7, 135.0, 151.3, 185.4. MS m/z: 294.3 (M⁺). IR (KBr) cm¹: 3289, 2929, 1649. UV ax (MeOH) nm (log ε): 254 (4.50), δ 14 (1.49).

1,5-Bis(ethanolamino)anthraquinone (IVb). The compound was synthesized as Example 1 and analyzed: 78% yield. mp 229-230° C. (EA/n-hexane). ¹H-NMR (CDCl₃) δ: 3.63 (4H, d, J=2.4 Hz), 4.06 (2H, s), 5.35 (4H, d, J=3.6 Hz), 7.54 (2H, d, J=8.4 Hz), 7.80 (2H, d, 0.1-7.2 Hz), 7.97 (2H, t, J=8.1 Hz), 10.16 (2H, br). ¹³C-NMR (DMSO-d) o: 49.99, 64.59, 117.26, 119.40, 122.32, 140.65, 140.74, 156.39, 185.36. MS m/z: 327.3 (M⁺). IR (KBr) cm⁻¹: 3370, 1646. UV λₘₐₓ (MeOH) nm (log ε): 521 (0.69).

1,5-Bis(isopropylamino)anthraquinone (IVc). The compound was synthesized as Example 1 and analyzed: 65% yield. mp 170-172° C. (EA/n-hexane). ¹H-NMR (CDCl₃) o: 1.39 (6H, d, J=6.0 Hz), 3.85-3.92 (2H, q), 7.02 (2H, d, J=3.3 Hz), 7.52 (2H, d, J=7.5 Hz), 7.57 (2H, t, J=3.7 Hz), 9.78 (2H, d, J=6 Hz). ¹³C-NMR (CDCl₃) 5:22.45, 43.30, 112.48, 114.05, 116.25, 134.68, 136.24, 150.29, 185.00. MS m/z: 323 (M⁺). IR (KBr) cm⁻¹: 3289, 1648. UV εₘₐₓ (MeOH) nm (log ε): 527 (0.33).

1,5-Bis(2-dimethylaminoethylamino)anthraquinone (IVd). The compound was synthesized as Example 1 and analyzed: 43% yield. mp 187-188° C. (EA/n-hexane).

¹H-NMR (CDCl₃) δ: 2.39 (12H, s), 2.71 (4H, q, J=6.4 Hz), 3.46-3.52 (4H, q, J=6.0 Hz), 7.00 (2H, d, J=8.4 Hz), 7.52-7.63 (4H, m), 9.80 (2H, br, s). ¹³C-NMR (DMSO-d) o: 41.02, 45.52, 58.12, 113.21, 114.87, 116.20, 135.05, 136.34, 151:18, 185.35. MS m/z: 381.1 (M⁺). IR (KBr) cm⁻¹: 3277, 1642. UV λₘₐₓ (MeOH) nm (log ε): 522 (0.42).

1,5-Bis[2-(2-Aminoethylamino)ethanol]anthraquinone (IVe). The compound was synthesized as Example 1 and analyzed: 62% yield. mp 160-161° C. (EA/n-hexane).

¹H-NMR (CDCl₃) δ: 2.67 (4H, d, J=5.0 Hz), 3.38 (8H, J J=6.0 Hz), 3.50 (2H, s), 4.54 (2H, s), 7.19 (2H, d, J=8.0 Hz), 7.44 (2H, t, J=6.0 Hz), 7.64 (2H, t, J=6.4 Hz), 9.79 (2H s). ³C-NMR (CDCl₃) o: 42.45, 48.00, 51.48, 60.54, 112.15, 114.31, 117.23, 135.60, 135.70, 151.18, 184.26. MS m/z: 413.1 (M⁺). IR (KBr) cm⁻¹: 3400, 3288, 1640, 1590. UV λₘₐₓ (MeOH) nm (log ε): 522 (1.07).

1,5-Bis(propylamino)anthraquinone (IVf). The compound was synthesized as Example 1 and analyzed: 73% yield. mp 153-154° C. (EA/n-hexane). ¹H-NMR (CDCl₃) δ: 1.15 (6H, t, J=7.5 Hz), 1.81-1.88 (4H, q, J=7.1 Hz), 3.32-3.38 (4H, q, J=6.4 Hz), 7.03 (2H, d, J=7.8 Hz), 7.56 (2H, t, J=3.7 Hz), 7.61 (2H, t, J=2.4 Hz), 9.79 (2H, br). ¹³C-NMR (CDCl₃) δ: 11.70, 22.27, 45.27, 113.33, 115.4, 117.22, 135.21, 136.20, 150.82, 185.34. MS m/z: 323 (M⁺). IR (KBr) cm¹: 3330, 1640. UV λₘₐₓ (MeOH) nm (log ε): 524 (0.16).

1,5-Bis(butylamino)anthraquinone (IVg). The compound was synthesized as Example 1 and analyzed: 72% yield. mp 152-153° C. (EA/n-hexane). ¹H-NMR (CDCl₃) δ: 1.02-1.07 (6H, q), 1.50-1.60 (4H, m), 1.74-1.84 (4H, m), 3.33-3.39 (4H, q), 7.01 (2H, d, J=7.5 Hz), 7.54 (2H, t, J=9.0 Hz), 7.57-7.60 (2H, q, J=3.0 Hz). 9.79-9.77 (2H, d, J=6.0 Hz).

¹³C-NMR (CDCl₃). 13.75, 20.32, 31.22, 42.62, 112.92, 114.55, 116.25, 135.05, 136.38, 151.54, 185.41. MS m/z: 350.3 (M⁺), 307.2. IR (KBr) cm⁻¹: 3299, 2947, 1623, 1500. UV λₘₐₓ (MeOH) nm (log ε): 527 (0.25).

1,5-Bis(propanolamino)anthraquinone (IVh). The compound was synthesized as Example 1 and analyzed: 74% yield. mp 178-180° C. (EA/n-hexane). ¹H-NMR (DMSO) δ: 1.74-1.83 (4H, m), 3.37 (4H, t, J=6.6 Hz), 3.51 (4H, q, J=5.6 Hz), 4.63 (2H, t, J=4.6 Hz), 7.12 (2H, d, J=8.7 Hz), 7.38 (2H, d, J=7.5 Hz), 7.57 (2H, t, J 7.9 Hz). 9.65 (2H, t, J 5.2 Hz). ¹³C-NMR (DMSO): 31.96, 38.35, 58.41, 112.08, 114.33, 117.10, 135.55, 135.66, 151.27, 184.37. MS m/z: 354 (M⁺). IR (KBr) cm⁻¹: 3359, 1624. UV λₘₐₓ (MeOH) nm (log ε): 522 (0.41), 283 (0.41).

1,5-Bis(cyclopentylamino)anthraquinone (IVi). The compound was synthesized as Example 1 and analyzed: 55% yield. mp 198-201° C. (EA/n-hexane). ¹H-NMR (CDCl₃) δ: 1.72-1.78 (8H, m), 1.86 (4H, t, J=5.7 Hz), 2.13 (4H, t, J=5.8 Hz), 4.03 (2H, d, J=4.8 Hz), 7.03-7.06 (2H, q, J=3.2 Hz), 7.52 (2H, d, J=7.5 Hz), 7.57 (2H, t, J=3.7 Hz), 9.85 (2H, d, J=5.4 Hz). ¹³C-NMR (CDCl₃) δ: 24.08, 33.5.8, 53.88, 112.92, 114.46, 117.10, 134.89, 136.44, 151, 185.29. MS m/z: 375.2 (M⁺), 307, 154, 136. IR (KBr) cm⁻¹: 3388; 1640. UV λₘₐₓ (MeOH) nm (log F: 528 (0.39).

1,5-Bis(butylamino)anthraquinone (IVj). The compound was synthesized as Example 1 and analyzed: 72% yield. m.p. 152-153° C. (EA/n-hexane). ¹H-NMR (CDCl₃) δ: 1.02 (6H, q, J=4.9 Hz), 1.50-1.60 (4H, m), 1.74-1.84 (4H, m), 3.33 (4H, q, J-6.4 Hz), 7.00 (2H, d, J=7.5 Hz), 7.52 (2H, t, J=9.0 Hz), 7.57 (2H, q, J=3.0 Hz). 9.79 (2H, br). ³C-NMR (CDCl₃) o: 13.75, 20.32, 31.22, 42.62, 112.92, 114.55, 116.25, 135.05, 136.38, 151.54, 185.41. IR (KBr) cm⁻¹: 3299, 2947, 1623, 1500 UV λₘₐₓ (MeOH) nm (log ε): 527 (0.25), 283 (0.19). MS m/z: 350 (M⁺), 307.

1,5-Bis(butanolamino-)-anthraquinone (IVk). The compound was synthesized as Example 1 and analyzed: 65% yield. m.p. 120-122° C. (EA/n-hexane). ¹H-NMR (DMSO) δ: 1.78 (4H, q, J=4.6 Hz), 1.86 (4H, q, J=4.3 Hz), 3.39 (4H, t, J=6.6 Hz), 3.76 (4H, t, J=6.1 Hz), 4.18 (2H, s), 7.00 (2H, d, J=6.3 Hz), 7.52-7.61 (4H, m), 9.77 (2H, br). ¹³C-NMR (DMSO) δ: 25.50, 30.10, 60.58, 63.60, 112.08, 114.38, 117.20, 135.55, 135.69, 151.26, 184.42. IR (KBr) cm⁻¹: 3384, 1643. UV λₘₐₓ (MeOH) nm (log ε): 523 (1.30), 284 (1.10). MS m/z: 382 (M⁺).

1,5-Bis(aminobutylamino)-anthraquinone (IVl). The compound was synthesized as Example 1 and analyzed: 65% yield. m.p. 208-210 (EA/n-hexane).? ¹H-NMR (CDCl₃) δ: 1.66-1.73 (4H, q, J=10.6 Hz), 1.82 (4H, q, J=6.3 Hz), 2.73-2.83 (4H, m), 2.84 (4H, t; J 3.4 Hz), 3.36 (4H, t, J=6.3 Hz), 6.99 (2H, d, J=7.5 Hz), 7.52-7.60 (4H, m), 9.76 (2H, s). IR (KBr) cm⁻¹: 3282, 1641, 1594. UV λₘₐₓ (MeOH) nm (log ε): 514 (1.17), 280 (1.02). MS m/z: 380 (M⁺).

1,5-Bis(aminopentylamino)-anthraquinone (IVm). The compound was synthesized as Example 1 and analyzed: 40% yield. m.p. 113-115 (EA/n-hexane). ¹H-NMR (CDCl₃) δ: 0.87 (4H, t, J=7.6 Hz), 1.56 (8H, d, J=3.3 Hz), 1.80 (4H, d, J=7.5 Hz), 2.75 (4H, t, J=6.6 Hz), 3.34 (4H, q, J=6.3 Hz), 6.98 (2H, q, J=3.1 Hz), 7.52-7.59 (4H, m), 9.75 (2H, s). IR (KBr) cm⁻¹: 3285, 1646. UV λₘₐₓ (MeOH) nm (log ε): 524 (0.47), 283 (0.45). MS m/z: 408 (M⁺).

1,5-Bis(hexylamino)-anthraquinone (IVn). The compound was synthesized as Example 1 and analyzed: 65% yield. m.p. 142-144° C. (EA/n-hexane). ¹H-NMR (CDCl₃) δ: 0.95 (6H, t, J=3.5 Hz), 1.37 (4H, q, J 3.5 Hz), 1.47 (4H, t, J=7.2 Hz), 1.75 (4H, t, J=7.3 Hz), 1.82 (4H, d, J=6.9 Hz) 3.32 (4H, t, J=5.4 Hz), 6.99 (2H, d, J=6.0 Hz), 7.52 (2H, t, J=7.5 Hz), 7.58 (2H, q, J=2.2 Hz). 9.74 (2H, br). ¹³C-NMR (CDCl₃) δ: 13.92, 22.50, 26.75, 29.29, 31.52, 42.97, 114.53, 116.23, 127.86, 135.03, 136.38, 151.51, 185.39. IR (KBr) cm⁻¹: 3388, 1626. UV λₘₐₓ (MeOH) nm (log ε): 518 (0.15). MS m/z: 406 (M⁺).

1,5-Bis(cyclopentaneamino)-anthraquinone (IVo). The compound was synthesized as Example 1 and analyzed: 55% yield. m.p. 198-200° C. (EA/n-hexane).

¹H-NMR (CDCl₃) 1.72-1.78 (8H, m), 1.84 (4H, t, J=5.7 Hz), 2.11 (4H, t, J=5.8 Hz), 4.04 (2H, d, J=4.8 Hz), 7.03 (2H, q, J=3.2 Hz), 7.51 (2H, d, J-7.5 Hz), 7.56 (2H, t J=3.7 Hz), 9.86 (2H, d, J-5.4 Hz). ³C-NMR (CDCl₃) δ: 24.08, 33.58, 53.88, 112.92, 114.46, 117.10, 134.89, 136.44, 151.0, 185.29. IR (KBr) cm⁻¹: 3388, 1640. UV λₘₐₓ (MeOH) nm (log ε): 528 (0.39), 285 (0.30). MS m/z: 374 (M⁺).

1,5-Bis(2,3-dimethylcyclohexylamino)-anthraquinone (IVp). The compound was synthesized as Example 1 and analyzed: 46% yield. m.p. 188-190° C. (EA/n-hexane).

¹H-NMR (CDCl₃) 6:2.00 (12H, s), 2.14-3.67 (16H, m), 3.90 (2H, s), 7.00-9.99 (6H, m), 10.20 (2H, br). ¹³C-NMR (CDCl₃) 8:17.05, 20.72, 25.09, 33.61, 35.11, 38.57, 45.44, 57.46, 114.28, 116.50, 116.83, 134.97, 136.67, 151.57, 185.31. R (KBr) cm⁻¹: 3273, 1676. UV λₘₐₓ (MeOH) nm (log ε): 527 (0.47), 281 (0.36). MS m/z: 458 (M⁺).

1,5-Bis(phenolamino)-anthraquinone (IVq). The compound was synthesized as Example 1 and analyzed: 56% yield. m.p. 211-212° C. (EA/n-hexane). ¹H-NMR (CDCl₃) δ: 4.90 (4H, s), 6.92 (2H, d, J=8.7 Hz), 7.21 (4H, t), 7.48 (2H, t, J=8.1 Hz), 7.65-7.84 (2H, m), 8:30-8.38 (2H, m), 11.07 (2H, s). IR (KBr) cm⁻¹: 3420; 3323, 1642. UV max (MeOH) nm (log ε): 517 (0.28), 252 (1.49). MS m/z: 422 (M⁺).

1,5-Bis(benzylamino)-anthraquinone (IVr). The compound was synthesized as Example 1 and analyzed: 75% yield. m.p. 218-220° C. (EA/n-hexane). ¹H-NMR (CDCl₃) δ: 4.61 (4H, d, J=6.0 Hz), 6.95 (2H, d, J=8.4 Hz), 7.31-7.40 (6H, m), 7.41 (2H, t, J1=3.9 Hz), 7.63 (2H, d, J=3.0 Hz). 10.15 (2H, br). ³C-NMR (CDCl₃) δ: 47.0, 115.27, 116.84, 127.0, 127.31, 128.74, 135.12, 136.24, 138.12, 151.21, 185.38. IR (KBr) cm⁻¹: 3270, 1640. UV λₘₐₓ (MeOH) nm (log ε): 518 (0.59), 281 (0.71). MS m/z: 418 (M⁺), 347.

1,5-Bis(phenylethylamino)-anthraquinone (Ivs). The compound was synthesized as Example 1 and analyzed: 64% yield. m.p. 205-207° C. (EA/n-hexane). ¹H-NMR (CDCl₃) δ: 3.07 (211, t, J=7.5 Hz), 3.58-3.65 (2H, m, J=5.1 Hz), 7.00 (2H, d, J=8.1 Hz), 7.31 (2H, t, J=6.0 Hz), 7.36-7.41 (4H, m), 7.55 (2H, t, J-5.3 Hz), 7.59 (2H, d, J15.7 Hz). 9.83 (2H, br). ³C-NMR (CDCl₃) δ: 35.71, 44.61, 113.13, 114.88, 116.20, 126.52, 128.69, 135.07, 136.32, 138.87, 151.15, 185.36. IR (KBr) cm⁻¹: 3270, 1640. UV λₘₐₓ (MeOH) nm (log ε): 527 (0.13), 285 (0.28). MS m/z: 446 (M⁺).

1,8-Bis(ethylamine)-anthraquinone (Va). The compound was synthesized as Example 1 and analyzed: yield 46%. mp ₁52-154° C. UV λₘₐₓ (MeOH) nm (log ε): 522 (0.78). MS (FAB): 285 (88), 294.1 (M⁺). ¹H-NMR (300 MHz, CDCl₃) 6 (ppm): 1.42 (t, J=7.2 Hz, 6H), 3.39-3.48 (m, 4H), 7.12 (d. J5.1 Hz, 2H), 7.48-7.62 (m, 4H), 9.60 (s, 21H, NH). ¹³C-NMR (500 MHz, CDCl₃) 6 (ppm): 188.91, 182.96, 151.35, 137.89, 134.41, 126.32, 118.16, 114.93, 37.60, 14.50.

1,8-Bis(propylamine)-anthraquinone (Vb). The compound was synthesized as Example 1 and analyzed: yield 83%. mp 158-160° C. UV λₘₐₓ (MeOH) nm (log ε): 282 (0.87), 554 (0.80). MS (FAB): 293.2 (30), 322.3 (M⁺). ¹H-NMR (300 MHz, CDCl₃) δ (ppm): 1.10 (t, J=7.35 Hz, 6H), 1.78-1.90 (m, 4H), 3.29 (q, J6.3 Hz, 4H), 7.04 (d, J=8.1 Hz, 2H), 7.47-7.58 (m, 4H), 9.67 (s, 2H, NH). ³C-NMR (500 MHz, CDCl₃) 6 (ppm): 189.03, 184.75, 151.24, 134.37, 134.04, 117.65, 114.74, 114.34, 44.85, 22.43, 11.76.

1,8-Bis(isopropylamine)-anthraquinone (Vf). The compound was synthesized as Example 1 and analyzed: yield: 64%. mp 198-200° C. MS(APCI): 323.1(M, 100), 324.1(24). ¹H-NMR (300 MHz, CDCl₃) 6 (ppm): 1.40-1.43 (q, J=²5 Hz, 12H, CH₃), 3.86-3.92 (q, J=6.3 Hz, 2H, N—CH), 7.06-7.09 (d, J=8.4 Hz, 2H), 7.47-7.57 (m, J=7.8 Hz, 4H), 9.64 (s, 2H, NH)

¹³C-NMR (300 MHz, CDCl₃)₆ (ppm): 185.00, 150.29, 136.24, 134.68, 116.25, 114.05, 112.48, 43.30, 22.45.

1,8-Bis(aminopentylamino)-anthraquinone (V_{L}). The compound was synthesized as Example 1 and analyzed: yield 35%. mp 115-116° C. UV λₘₐₓ (MeOH) nm (log ε): 281 (1.89), 545 (1.72). MS (APCI): 409.1 (Mt), 410.2 (20) ¹H-NMR (300 MHz, CDCl₃) 6 (ppm): 0.86 (t, J=7.8 Hz, 4H), 1.48 (t, J=6.6 Hz, 4H), 1.57 (t, J=3.3 Hz, 4H), 1.81 (t, J=6.6 Hz, 4H), 2.71-2.79 (m, J 5.9 Hz, 4H), 3.33 (q, J 6.5 Hz, 4H), 7:03 (d, J=8.7 Hz, 2H), 7.47-7.58 (m, J=7.9 Hz, 4H), 9.65 (s, 2H, NH).

1,4-Bis(chloroacetamido)-9,10-anthracenedione (VI₁). The compound was synthesized as Example 1 and analyzed: yield 77%. m.p. 285-287° C. (chloroform). ¹H-NMR (DMSO) δ: 4.60 (4H, s), 8.00-7.97 (2H, m), 8.27-8.24 (2H, m), 8.99 (2H, s), 12.80 (2H, s). IR (KBr) cm⁻¹: 1595, 1650. UV λₘₐₓ (MeOH) nm (log ε): 486.0 (1.52). MS m/z: 390 (M⁺) Anal. Calcd. for C₁₈H₁₀ClN₂O₄: C, 55.26; H, 3.09. Found: C, 55.10; H, 3.00.

1,4-Bis(2-chlorobenzoylamido)-9,10-anthracenedione (VI₂). The compound was synthesized as Example 1 and analyzed: 82% yield. m.p. 324-326° C. (EA/n-hexane). ¹H-NMR (CDCl₃) δ: 7.46-7.54 (4H, m), 7.55-7.60 (4H, m), 7.78-7.86 (2H, m), 8.28-8.31 (2H, m), 9.46 (2H, s), 13.05 (2H, s). ³C-NMR (CDCl₃) δ: 117.17, 127.14, 127.17, 129.27; 129.45, 131.65, 131.72, 134.46, 138.37, 166.1, 186.1. IR (KBr) cm⁻¹: 3365, 1655. UV λₘₐₓ (MeOH) nm (log ε): 365.0 (1.18). MS m/z: 514 (M⁺), 516.

1,4-Bisacetamido-9,10-anthracenedione (VI₃). The compound was synthesized as Example 1 and analyzed: 92% yield. m.p. 278-280° C. (EA/n-hexane). ¹H-NMR (CDCl₃) 2.38 (6H, s), 7.85-7.88 (2H, m), 8.31-8.34 (2H, m), 9.20 (2H, s), 12.56 (2H, s), ¹³C-NMR (CDCl₃) δ: 25.70, 116.05, 127.02, 129.05, 133.33, 134.36, 138.49, 169.71, 186.88 IR (KBr) cm⁻¹: 3370, 1610. UV λₘₐₓ (MeOH) nm (log ε): 465.0 (0.25). MS m/z: 280 (M⁺).

1,4-Bisbenzoylamido-9,10-anthracenedione (VI₄). The compound was synthesized as Example 1 and analyzed: 83% yield. m.p. 285-287° C. (EA/n-hexane).

¹H-NMR (CDCl₃) δ: 7.64-7.69 (6H, m), 7.89-7.92 (2H, m), 8.23-8.26 (4H, d, J=2.1 Hz), 8.41-8.44 (2H, m), 9.52 (2H, s), 13.62 (2H, s). IR (KBr) cm⁻¹: 3340, 1635. UV λₘₐₓ (MeOH) nm (log ε): 486.0 (1.76). MS m/z: 446.1 (M⁺), 447.1.

1,4-Bis(3-chlorobenzoylamido)-9,10-anthracenedione (V15). The compound was synthesized as Example 1 and analyzed: 85% yield. m.p. 244-246° C. (EA/n-hexane).

¹H-NMR (CDCl₃) δ: 7.54-7.64 (4H, m), 7.87-7.90 (2H, m), 8.17 (2H, s), 8.37-8.40 (2H, m), 9.41 (2H, s), 13.56 (2H, s). ¹³C-NMR(CDCl₃) δ: 117.28, 125.45, 127.34, 128.18, 129.30, 130.11, 132.30, 133.23, 136.35, 138.82, 164.96, 187.22. IR (KBr) cm⁻¹: 3380, 1665. UV λₘₐₓ (MeOH) nm (log ε): 486.0 (1.60). MS m/z: 514.0 (M⁺), 516.

1,4-Bis(3-methylbenzoylamido)-9,10-anthracenedione (VI₆). The compound was synthesized as Example 1 and analyzed: 82% yield. m.p. 242-244° C. (EA/n-hexane).

¹H-NMR (CDCl₃) δ: 2.57 (6H, s), 7.47-7.56 (6H, m), 7.87-7.90 (2H, m), 8.01 (2H, s, J=6.3 Hz), 8.39-8.42 (2H, m), 9.48 (2H, s), 13.54 (2H, s). IR (KBr) cm⁻¹: 3335, 1620. UV λₘₐₓ (MeOH) nm (log ε): 352 (2.33). MS m/z: 474.1 (M⁺), 475.1.

1,4-Bis(3-chloropropionamido)-9,10-anthracenedione (V17). The compound was synthesized as Example 1 and analyzed: yield 78%. m.p. 230-231° C. (EA/n-hexane).

¹H-NMR (CDCl₃) o: 3.0-3.04 (4H, t, J=6.6 Hz), 3.98 (4H, t, J=6.5 Hz), 7.70-7.93 (H, m), 8.35-8.32 (2H, m), 9.23 (2H, s), 12.72 (2H, s), ¹³C-NMR(CDCl₃) δ: 39.3, 41.52, 116.95, 127.17, 129.12, 133.23, 134.56, 138.12, 169.08, 186.98. MS m/z: 418.1 (M⁺). IR (KBr) cm⁻¹: 1600, 1650, 1710. UV λₘₐₓ (MeOH) nm (log ε): 465.0 (1.04). Anal. Calcd. for C₂₀H₁₆Cl₂N₂O₄: C, 57.30; H, 3.85. Found: C, 57.12; H, 3.55.

1,4-Bis(2,4-dichlorobenzoylamido)-9,10-anthracenedione (VI₈). The compound was synthesized as Example 1 and analyzed: 80% yield. m.p. 331-333° C. (EA/n-hexane).

¹H-NMR (CDCl₃) δ: 7.47 (2H, d, J=9.3 Hz), 7.60 (2H, s), 7.73-7.76 (2H, m), 7.86 (2H, d, J=6.9 Hz), 8.28-8.31 (2H, m), 9.42 (2H, s), 13.09 (2H, s). IR (KBr) cm⁻¹: 3340, 1635. UV λₘₐₓ (MeOH) nm (log ε): 519.0 (0.07). MS m/z: 582.0 (M⁺), 584.0.

1,4-Bis(2-chloropropionamido)-9,10-anthracenedione (VI₉). The compound was synthesized as Example 1 and analyzed: 76% yield m.p. 233-235° t. (EA/n-hexane).

¹H-NMR (CDCl₃) o: 1.93 (6H, d, J=7.2 Hz), 4.65 (2H, q, J=7.7 Hz), 7.6-7.89 (2H, m), 8.36-8.39 (2H, m), 9.21 (2H, s), 13.24 (2H, s).

¹³C-NMR(CDCl₃) δ: 22.44, 55.99, 118.10, 127.30, 128.71, 133.05, 134.55, 137.83 169.67, 186.76. IR (KBr) cm⁻¹: 3155, 1648. UV λₘₐₓ (MeOH) nm (log ε): 459.0 (0.51). MS m/z: 418 (M⁺), 355. Anal. Calcd. for C₂₀H₁₆Cl₂N₂O₄: C, 57.30; H. 3.85. Found: C, 57.09; H, 3.22.

1,4-Bis(2-fluorobenzoylamido)-9,10-anthracenedione (VI₁₀) The compound was synthesized as Example 1 and analyzed: 83% yield. m.p. 238-239° C. (EA/n-hexane).

¹H-NMR(CDCl₃) δ: 7.29-7.40 (4H, m), 7.58-7.65 (2H, m), 7.83-7.87 (2H, m), 8.15 (2H, t, J=3.5 Hz), 8.34-8.37 (2H, m), 9.41 (2H, s), 13.29-13.32 (2H, d, J=6.9 Hz). ¹³C-NMR(CDCl₃) δ: 16.35, 118.39, 122.85, 124.64, 127.11, 129.57, 131.70, 133.36, 133.59, 134.23, 138.19, 160.88, 163.09, 186.60. IR (KBr) cm⁻¹: 3375, 1660. UV λₘₐₓ (MeOH) nm (log E: 466.0 (1.09). MS m/z: 482.2 (M⁺), 483.2.

1,4-Bis(2-nitrobenzoylamido)-9,10-anthracenedione (VI₁₁). The compound was synthesized as Example 1 and analyzed: 70% yield. m.p. 349-351° C. (EA/n-hexane).

¹H-NMR (CDCl₃) o: 7.87-7.93 (4H, m), 7.94-8.04 (6H, m), 8.06-8.24 (4H, m), 9.00 (2H, s), 12.65 (2H, s). IR (KBr) cm⁻¹: 3355, 1645. UV λₘₐₓ (MeOH) nm (log ε): 437.0 (1.42). MS m/z: 536 (M⁺), 537.

1,4-Bis(3-fluorobenzoylamido)-9,10-anthracenedione (VI₁₂). The compound was synthesized as Example 1 and analyzed: 75% yield. m.p. 273-275° C. (EA/n-hexane).

¹H-NMR (CDCl₃) δ: 7.37 (2H, t, J=3.8 Hz), 7.59-7.66 (4H, m), 7.89-7.92 (2H, m), 8.01 (2H, d, J=7.5 Hz), 8.40-8.43 (2H, m), 9.47 (2H, s), 13.61 (2H, s). ¹³C-NMR (CDCl₃) δ: 114.87, 115.17, 119:15, 123.07, 127.3.1, 129.30, 130.44, 133.27, 134.58, 135.23, 138.85, 162.88, 165.05, 187.25. IR (KBr) cm⁻¹: 3370, 1655. UV λₘₐₓ (MeOH) nm (log ε): 521.0 (1.08). MS m/z: 482.1 (M⁺), 483.1.

1,4-Bis(2,4,6-trichlorobenzoylamido)-9,10-anthracenedione (VI₁₃). The compound was synthesized as Example 1 and analyzed: 60% yield. m.p. 361-363° C. (EA/n-hexane). ¹H-NMR (CDCl₃) o: 7.33-7.41 (2H, m), 7.85-7.91 (2H, m), 8.09 (2H, d, J=7.8 Hz), 8.19 (2H, d, J=7.5 Hz), 10.33 (2H, s). IR (KBr) cm¹: 3355, 1675. UV λₘₐₓ (MeOH) nm (log ε): 479.0 (0.43). MS m/z: 653.16 (M⁺).

1,4-Bis(2,3,6-trifluorobenzoylamido)-9,10-anthracenedione (VI₁₄). The compound was synthesized as Example 1 and analyzed: 84% yield. m.p. 345-347° C. (EA/n-hexane). ¹H-NMR (CDCl₃) δ: 7.08 (2H, t, J=8.9 Hz), 7.32-7.42 (2H, m), 7.85-7.88 (2H, m), 8.29-8.32 (2H, m), 9.43 (2H, s), 13.18 (2H, s). ³C-NMR (CDCl₃) δ: 109.11, 112.11, 117.73, 17.2, 129.22, 133.09, 134.69, 138.02, 144.80, 158.44, 149.50, 165.05, 186.95. IR (KBr) cm⁻¹: 3370, 1610. UV λₘₐₓ (MeOH) nm (log ε): 463.0 (1.40). MS m/z: 553.9 (Mt).

1,4-Bis(2,4,5-trifluorobenzoylamido)-9,10-anthracenedione (VI₁₅). The compound was synthesized as Example 1 and analyzed: 70% yield. m.p. 310-312° C. (EA/n-hexane). ¹H-NMR (CDCl₃) δ: 7.14-7.19 (2H, m), 7.88 (2H, d, J=7.0 Hz), 8.02-8.04 (2H, m), 8.35 (2H, d, J=8.0 Hz), 9.36 (2H, s), 13.32 (2H, s). IR (KBr) cm⁻¹: 3340, 1620. UV λₘₐₓ (MeOH) nm (log ε): 486.0 (0.84). MS m/z: 554.0 (M⁺).

1,4-Bis(4-chlorobenzoylamido)-9,10-anthracenedione (VI 16). The compound was synthesized as Example 1 and analyzed: 84% yield. m.p. 320-322° C. (EA/n-hexane).

¹H-NMR (CDCl₃) δ: 7.59-7.62 (4H, d, J=8.7 Hz), 7.89-7.92 (2H, m), 8.16 (4H, d, J=8.1 Hz), 8.39-8.42 (2H, m), 9.48 (2H, s), 13.61 (2H, s). ³C-NMR (CDCl₃) δ 116.35, 118.39, 122.85, 124.64, 127.11, 129.57, 131.70, 133.36, 133.59, 134.23, 138.19, 160.88, 163.09, 186.60. IR (KBr) cm⁻¹: 3400, 1690. UV λₘₐₓ (MeOH) nm (log ε): δ 17.0 (0.34). MS m/z: 514.0 (M⁺), 516.0.

1,4-Bis(cyclohexaneamido)-9,10-anthracenedione (VI₁₇)

The compound was synthesized as Example 1 and analyzed: 82% yield. m.p. 309-310° C. (EA/n-hexane). ¹H-NMR(CDCl₃) 1.91-1.95 (12H, m), 2.11-2.15 (8H, d), 2.42-2.52 (2H, m), 7.83-7.89 (2H, m), 8.33-8.36 (2H, m), 9.24 (2H, s), 12.61 (2H, s).

¹³C-NMR (CDCl₃) δ: 25.72, 29.64, 47.45, 116.66, 127.04, 129.27, 133.39, 134.22, 138.79, 176.08, 186.89 IR (KBr) cm⁻¹: 3365, 1649. UV λₘₐₓ (MeOH) nm (log ε): 470.0 (0.70). MS m/z: 458.1 (M⁺), 460.2.

1,4-Bis(2,4-difluorobenzoylamido)-9,10-anthracenedione (VI₁₈). The compound was synthesized as Example 1 and analyzed: 66% yield. m.p. 315-317° C. (EA/n-hexane).

¹H-NMR (CDCl₃) δ: 7.03-7.13 (2H, m), 7.85-7.88 (2H, m), 8.01 (2H, t, J=6.3 Hz), 8.34-8.37 (2H, m), 9.38 (2H, s), 13.30 (2H, s): IR (KBr) cm⁻¹: 3325, 1615. UV λₘₐₓ (MeOH) nm (log ε): 469.0 (1.31). MS m/z: 518.0 (M⁺), 520.0.

1,4-Bis(3-cyclopentanepropionamido)-9,10-anthracenedione (VI₁₉). The compound was synthesized as Example 1 and analyzed: 87% yield. m.p. 166-167° C. (EA/n-hexane). ¹H-NMR(CDCl₃) 1.67 (4H, d, J=5.1 Hz), 1.86-1.91 (10H, m) 2.60 (2H, t, J 7.5 Hz), 7.85-7.88 (2H, m), 8.33-8-0.36 (2H, m), 12.60 (2H, s). ¹³C-NMR (CDCl₃) δ: 5.14, 31.68, 32.50, 38.1, 39.69, 114.98, 127.03, 129.15, 133.38, 134.26, 138.60, 173.14, 186.87. IR (KBr) cm⁻¹: 3360, 1625. UV λₘₐₓ (MeOH) nm (log ε): 469.0 (038). MS m/z: 486.2 (M⁺), 487.2.

1,4-Bis(cyclopentaneamido)-9,110-anthracenedione (VI₂₀). The compound was synthesized as Example 1 and analyzed: 80% yield. m.p. 243-244° C. ((EA/n-hexane).

¹H-NMR (CDCl₃) 1.90-1.99 (8H, m), 2.00-2.17 (8H, m), 2.90-3.01 (2H, p, J=8.1 Hz), 9.22 (2H, s), 12.63 (2H, s). ¹³C-NMR (CDCl₃) δ: 25.88, 29.62, 30.37, 48.13, 116.52, 127:00, 129.18, 133.42, 134.17, 138.77; 176.09, 186.85. IR (KBr) cm⁻¹: 3375, 1645. UV λₘₐₓ (MeOH) nm (log ε): 474.0 (0.50). MS m/z: 430 (Mt.

1,4-Bis(cyclopropionamido)-9,10-anthracenedione (VI₂₁). The compound was synthesized as Example 1 and analyzed: 76% yield. m.p. 281-82° C. (EA/n-hexane). ¹H-NMR (CDCl₃) 0.98-1.02 (8H, m), 1.79-1.84 (2H, m), 7.85-7.88 (2H, m), 8.33-8.36 (2H, m), 9.19 (2H, s), 12.86 (2H, s). ¹³C-NMR (CDCl₃) δ: 8.51, 17.00, 116.26, 127.01, 129.22, 133.48, 134.22, 138.63, 173.40, 186.88. IR (KBr) cm⁻¹: 3315, 1680. UV λₘₐₓ (MeOH) nm (log ε): 490.0 (0.64). MS m/z: 374.3 (M⁺).

1,4-Bis(2-thiopheneamido)-9,10-anthracenedione (VI₂₂). The compound was synthesized as Example 1 and analyzed: 79% yield. m.p. 321-322° C. (EA/n-hexane).

¹H-NMR (CDCl₃) o: 7.26-7.29 (2H, t, J=3.1 Hz), 7.67-7.69 (2H, d, J=5.1 Hz), 7.88-7.91 (2H, m), 8.03 (2H, t, J=2.4 Hz), 8.39-8.42 (2H, m), 9.38 (2H, s), 13.61 (2H, s). IR (KBr) cm⁻¹: 3380, 1605. UV λₘₐₓ ((MeOH)) nm (log ε): 514.0(0.49). MS m/z: 458.0 (M⁺).

1,4-Bis(2,3-dichloro-5-trifluorobenzoylamido)-9,10-anthracenedione (VI₂₃). The compound was synthesized as Example 1 and analyzed: 77% yield. m.p. 321-322° C. (EA/n-hexane). ¹H-NMR (CDCl₃) o: 7.62 (2H, d, J=8.7 Hz), 7.67 (2H, d, J=6.6 Hz), 7.88-7.89 (2H, m), 8.30-8.32 (2H, m), 9.38 (2H, s), 13.13 (2H, s). IR (KBr) cm⁻¹: 3300, 1650. UV λₘₐₓ (MeOH) nm (log ε): 487.0 (0.87). MS m/z: 619.7 (M⁺), 620.7.

1,4-Bis(2-furoylamido)-9,10-anthracenedione (VI₂₄). The compound was synthesized as Example 1 and analyzed: 63% yield. m.p. 365-367° C. (EA/n-hexane).

¹H-NMR (CDCl₃) δ: 6.66-6.67 (2H, d), 7.39-7.40 (2H, d, J=3.6 Hz), 7.76 (2H, s), 7.87-7.90 (2H, m), 8.41-8.43 (2H, m), 9.42 (2H, s), 13.55 (2H, s). IR (KBr) cm⁻¹: 3366, 1658. UV λₘₐₓ (MeOH) nm (log ε): 578.0 (0.39). MS m/z: 46.8 (M⁺).

1,4-Bis(2-thiopheneacetamido)-9,10-anthracenedione (VI₂₅). The compound was synthesized as Example 1 and analyzed: 75% yield. m.p. 158-160° C. (EA/n-hexane).

¹H-NMR (CDCl₃) □□□□ (4H, s), 7.10-7.13 (2H, t, J=4.4 Hz), 7.17 (2H, d, J=3.3 Hz), 7.35 (2H, d, J=5.7 Hz), 7.81-7.84 (2H, m), 8.22-8.25 (2H, m), 9.19 (2H, s), 12.58 (H, s).

¹³C-NMR(CDCl₃)° C., 39.71, 117.23, 125.60, 127.05, 17.22, 127.68, 128.91, 133.14, 134.29, 134.97, 138.19, 169.67, 186.66. IR (KBr) cm⁻¹: 3375, 1610. UV λₘₐₓ (MeOH) nm (log ε): 486.56 (0.72). MS m/z: 486.1 (M⁺), 487.1.

1,4-Bis(2,5-dimethylfuran-3-carbonylamido)-9,10-anthracenedione (VI₂₆). The compound was synthesized as Example 1 and analyzed: 79% yield. m.p. 2847286° C. (EA/n-hexane). ¹H-NMR (CDCl₃) δ: 2.40 (6H, s), 2.69 (6H, s), 6.59 (2H, s), 7.87-7.84 (2H, m), 8.39-8.36 (2H, m), 9.37 (2H, s), 13.05 (2H, s). IR (KBr) cm⁻¹: 3310, 1645. UV λₘₐₓ (MeOH) nm (log ε): 582.0 (0.31). MS m/z: 482 (M⁺).

1,4-Bis(trans-2-phenyl-1-cyclopropaneamido)-9,10-anthracenedione (VI₂₇). The compound was synthesized as Example 1 and analyzed: 68% yield. m.p. 272-274° C. (EA/n-hexane). ¹H-NMR (CDCl₃) δ: 1.79-1.86 (2H, m, J=4.7 Hz), 2.03-2.09 (2H, m), 2.69-2.76 (4H, in, J=3.3 Hz), 7.22-7.40 (10H, m, J=6.9 Hz), 7.83-7.86 (2H, m), 8.29-8.32 (2H, m), 9.26 (2H, s), 12.93 (2H, s). ¹³C-NMR (CDCl₃) o: 16.59, 17.07, 28.59, 116.34, 126.24, 126.46, 127.02, 128.47, 129.14, 133.34, 134.32, 138.51, 140.1, 171.76, 186.85. IR (KBr) cm⁻¹: 3320, 1635. UV λₘₐₓ (MeOH) nm (log ε): 486.0 (0.74). MS m/z: 526.0 (M⁺).

1,4-Bis(phenylthioacetamido)-9,10-anthracenedione (VI₂₈). The compound was synthesized as Example 1 and analyzed: 84% yield. m.p. 137-139° C. (EA/n-hexane).

¹H-NMR (CDCl₃) δ: 3.91 (4H, s) 7.26 (6H, t, J=6.5 Hz), 7.50 (4H, d, J=4.5 Hz), 7.84-7.87 (2H, m), 9.13 (2H, s), 12.18 (2H, s) ¹³ C-NMR (CDCl₃) δ: 40.16, 117.75, 126.98, 127.11, 128.76, 129.12, 129.63, 133.22, 134.32, 134.55, 137.82, 158.59, 186.52. IR (KBr) cm⁻¹: 3345, 1670. UV λₘₐₓ (MeOH) nm (log ε): 485.0 (0.69). MS m/z: 538.0 (Mt), 539.1.

1,4-Bis(2,5-trifluorobenzoylamido)-9,10-anthracenedione (VI₂₉). The compound was synthesized as Example 1 and analyzed: 87% yield. m.p. 245-247° C. (EA/n-hexane). ¹H-NMR (CDCl₃) o: 7.17 (2H, d, J=9.6 Hz), 7.84-7.87 (2H, m), 7.96-8.01 (2H, t, J=8.0 Hz), 8.05 (2H, d, J=9.0 Hz), 9.38 (2H, s), 13.03 (2H, s). ¹³C-NMR (CDCl₃) δ: 112.40, 117.82, 120.64, 124.86, 125.57, 127.30, 127.72, 129.25, 130.44, 133.03, 134.29, 134.79, 136.88, 138.12, 165.26, 187.01. IR (KBr) cm⁻¹: 3355, 1655. UV λₘₐₓ (MeOH) nm (log ε): 524.0 (0.58). MS m/z: 718.2 (M⁺).

1,4-Bis(4-fluorobenzoylamido)-9,10-anthracenedione (VI₃₀). The compound was synthesized as Example 1 and analyzed: 88% yield. m.p. 309-311° C. (EA/n-hexane).

¹H-NMR (CDCl₃) o: 7.28-7.34 (4H, m, J=3.4 Hz), 7.89-7.92 (2H, m), 8.22-8.26 (4H, m, J=2.3 Hz), 8.39-8.42 (2H, m), 9.47 (2H, s), 13.59 (2H, s). ¹³C-NMR (CDCl₃) δ: 116.35, 118.39, 122.85, 124.64, 127.11, 129.57, 131.70, 133.36, 133.59, 134.23, 138.19, 160.88, 163.09, 186.60. IR (KBr) cm⁻¹: 3390, 1680. UV λₘₐₓ (MeOH) nm (log ε): 590.0 (0.09). MS m/z: 482.2 (M⁺), 483.2.

1,4-Bis(4-trifluorobenzoylamido)-9,10-anthracenedione (VI₃₁). The compound was synthesized as Example 1 and analyzed: 78% yield. m.p. 331-333° C. (EA/n-hexane).

¹H-NMR(CDCl₃) δ: 7.60 (4H, s), 7.91 (2H, d, J=8.1 Hz), 8.32-8.35 (4H, d, J=7.8 Hz), 8.42 (4H, m), 9.51 (2H, s), 13.70 (2H, s). ¹³C-NMR (CDCl₃) δ: 117.82, 120.08, 125.57, 127.30, 127.72, 129.25, 133.03, 134.29, 134.79, 136.88, 138.12, 165.26, 187.01. IR (KBr) cm⁻¹: 3335, 1645. UV λₘₐₓ (MeOH) nm (log ε): 518.0 (0.35). MS m/z: 582.0 (M⁺), 583.0.

1,4-Bis(4-fluorophenylacetamido)-9,10-anthracenedione (VI₃₂). The compound was synthesized as Example 1 and analyzed: 85% yield. m.p. 228-230° C. (EA/n-hexane).

¹H-NMR (CDCl₃) δ: 3.85 (4H, s), 7.14 (4H, t, J=4.0 Hz), 7.41-7.46 (4H, m), 7.82-7.85 (2H, m), 8.21-8.24 (2H, m), 9.16 (2H, s), 12.54 (2H, s). ¹³C-NMR (CDCl₃) δ: 45.09, 115.62, 117.01, 127.04, 128.88, 129.79, 131.09, 133.13, 134.36, 138.31, 161.20, 170.62, 186.72. IR (KBr) cm⁻¹: 3350, 1655. UV λₘₐₓ (MeOH) nm (log ε): 485.0 (0.64). MS m/z: 510.0 (M⁺).

1,4-Bis[2-(diethylamino)acetamido]-9,10-anthracenedione (VI₃₃). The compound was synthesized as Example 1 and analyzed: 50% yield. m.p. 85-86° C. (EA/n-hexane). ¹H-NMR (CDCl₃) δ: 1.55 (12H, t), 3.08-3.05 (8H, s), 4.32-4.29 (4H, s), 7.90-7.87 (2H, m), 7.52-7.63 (4H, m), 9.22 (2H, s).13.28 (2H, s). IR (KBr) cm⁻¹: 3365, 1688. UV λₘₐₓ (MeOH) nm (log ε): 525.0 (0.50). Anal. Calcd for C₂₆H₃₂N₄O₄, C, 67.22, H, 6.94.

Found: C, 67.02; H, 6.25.

1,4-Bis[3-(diethylamino)propionamido]-9,10-anthracenedione (VI₃₄). The compound was synthesized as Example 1 and analyzed: 40% yield. m.p. 169-171° C. (EA/n-hexane). ¹H-NMR (CDCl₃) δ: 2.80-2.76 (12H, t), 3.12-3.04 (8H, m), 3.25-3.21 (8H, m), 7.86 (2H, m), 8.23 (2H, m), 9.01 (2H, s), 12.68 (2H, br). IR (KBr) cm¹: 3160, 1636. UV λₘₐₓ (MeOH) nm (log ε): 485.0 (0.57).

1,4-Bis[2-(diethylamino)propionamido]-9,10-anthracenedione (VI₃₅) The compound was synthesized as Example 1 and analyzed: 75% yield. m.p. 175-177° C. (EA/n-hexane). ¹H-NMR (CDCl₃) 6:1.53-1.49 (12H, t), 1.94-1.90 (8H, m), 3.08-3.06 (6H, d), 4.66-4.59 (2H, m), 7.86-7.76 (2H, m), 8.37-8.32 (2H, m), 8.96-8.93 (2H, m), 13.23 (2H, br). IR (KBr) cm⁻¹: 3390, 1645. UV λₘₐₓ (MeOH) nm (log ε): 546 (0.9).

1,4-Bis[2-(aminomnethyl)cyclopropanepropionamido]-9,10-anthracenedione (VI₃₆). The compound was synthesized as Example 1 and analyzed: 63% yield. m.p. 172-174° C. (EA/n-hexane). ¹H-NMR (CDCl₃: 0.89 (10H, t, J=7.8 Hz), 10.90-1.94 (10H, m), 4.61-4.66 (2H, m), 7.78-7.88 (2H, m), 8.32-8.38 (2H, m), 8.93-8.96 (2H, d), 13.24 (2H, brs). IR (KBr) cm⁻¹: 3170, 1660. UV λₘₐₓ (MeOH) nm (log ε): 489.0 (0.72). MS m/z: 488.4 (M⁺), 307.2.

1,4-Bis[3-(aminomethyl)cyclopropanepropionamido]-9,10-anthracenedione (VI₃₇). The compound was synthesized as Example 1 and analyzed: 63% yield. m.p. 208-210° C. (EA/n-hexane). ¹H-NMR (CDCl₃) o: 0.91 (110H, t, J=6.0 Hz), 1.92 (4H, t, J=6.6 Hz), 4.59-4.66 (10H; m), 7.77-7.86 (2H, m), 8.33-8.38 (2H, m), 8.96 (2H, d, J=9.6 Hz). 13.23 (2H, br). IR (KBr) cm⁻¹: 3325, 1650. UV λₘₐₓ (MeOH) nm (log ε): 525.0 (0.29). MS m/z: 488.8 (M⁺).

1,5-bis-(2-chloroproplonamido)-9,10-anthraquinone (VIIa). The compound was synthesized as Example 1 and analyzed: 48% yield. mp 419.3° C. (EA/n-hexane). ¹H-NMR (CDCl₃) δ: 3.01 (t, 2H, J=6.3 Hz), 3.92 (t, 2H, J=6.6 Hz), 7.80 (t, 2H, J=8.1 Hz), 8.06 (d, 2H, J=7.5 Hz), 9.15 (d, 2H, J=8.4 Hz), 12.39 (s, 2H).

1,5-bis-(methylacetamido)-9,10-anthracenedione (VIIb). The compound was synthesized as Example 1 and analyzed: 45% yield. mp 321° C. (EA/n-hexane).

¹H-NMR(CDCl₃) 6:2.33 (s, 6H), 7.77 (t, 2H, J 8.7 Hz), 8.03 (d, 2H, J=7.8 Hz), 9.12 (d, 2H, J 8.7 Hz), 12.25 (s, 2H). ³C-NMR(CDCl₃) 5:25.70; 116.96, 122.48, 126.15, 134.57, 135.90, 142.04, 169.89, 186.61.

1,5-bis-[3-(methyl)benzoylamido)-9,10-anthracenedione (VIIc). The compound was synthesized as Example 1 and analyzed: 63% yield. mp 290.8° C. (EA/n-hexane).

¹H-NMR(CDCl₃) δ: 2.55 (s, 6H), 7.48-7.52 (m, 4H), 7.89 (t, 2H, J=7.5 Hz), 7.99 (d, 4H, J=6 Hz), 8.190 (d, 2H, J 6.3 Hz), 9.40 (d, 2H, J=8.7 Hz), 13.24 (s, 2H). ¹³C-NMR(CDCl₃) δ: 21.51, 122.84, 124.59, 126.44, 128.53, 128.84, 133.19, 134.51, 136.02, 138.86, 166.83, 186.91.

1,5-bis-(2-chloropropionamido)-9,10-anthracenedione (VIId). The compound was synthesized as Example 1 and analyzed: 70% yield. mp 288-289° C. (EA/n-hexane).

¹H-NMR(CDCl₃) δ: 1.87 (d, 3H), 4.60 (q, 1H, J=6.9 Hz), 7.81 (t, 2H, J=8.1 Hz), 8.14 (d, 2H, J 6.9 Hz), 9.12 (d, 21H, J=8.7 Hz), 12.94 (s, 21H).

1,5-bis-(2-chloroacetamidoamido)-9,10-anthracenedione (VIIe). The compound was synthesized as Example 1 and analyzed: 65% yield. mp 370° C. (EA/n-hexane). ¹H-NMR(CDCl₃) δ: 4.33 (s, 2H), 7.80 (t, 2H, J-8.1 Hz), 8.15 (d, 2H, J=6.9 Hz), 9.12 (d, 2H, J 8.7 Hz), 11.70 (s, 2H).

### Example 3

### Cytotoxicity Assay

Cytotoxic evaluations (XTT colorimetric assay). Tumor cell lines used were rat glioma C6 cells and human hepatoma G2 cells. The cells (2.5×10⁴ cells/ml) were placed into 96-well plates and preincubated for 24 to 72 h in complete medium. The drug concentration inhibiting 50% of cellular growth (IC₅₀, mg/ml) was determined using the XTT assay following 72 h of drug exposure. The results are the means of at least three independent experiments unless otherwise indicated. The results of this assay are provided in Table.

### Example 4

### Lipid Peroxidation

Fresh S.D. rat brains were obtained and the residual vessels were cleaned up. The fresh brains were then homogenized with Kreb's buffer. After centrifugation, the upper solution (about 9 ml) was obtained. Separate the 9 ml of solution'to about 18 vials (500 ml/vial), which are separated into control and experimental sets. Then add Kreb's buffer: (60 μl) and DMSO solution dissolved tested compounds (30 μl) respectively to the vials. After 10 minutes, add ferrous sulfate solution to the control and experimental sets and remain steady in 37° C. water bath. After 30 minutes, leave vials from the water bath and add trichloroacetic acid 10 ml (4% (w/v) in 0.3 N HCl) to denature the residual protein. Add 2-thiobarbituric acid solution 200 ml (0.5% (w/v) 2-thiobarbituric acid in 50% (v/v) acetic acid) to the solution and keep in 100° C. water bath for 15 minutes. The effects of tested compounds to lipid peroxidation are determined by UV to detect the percentages of red-colored product form by 2-thiobarbituric acid and malondialdehyde, which is one of the products formed by lipid peroxidation. The results of this assay are provided in Table.

### Example 5

### Cytotoxicity Assay

The tetrazolium reagent (MTT; 3-(4,5-di-methylthiazol)-2,5-diphenyl tetrazolium bromide, USB) was designed to yield a colored formazan upon metabolic reduction by viable cells. Approximately 2×10 cells were plated onto each well of a 96-well plate and incubated in 5% CO₂ at 37° C. for 24 h. To assess the in vitro cytotoxicity, each compound was dissolved in DMSO and prepared immediately before the experiments and was diluted into the complete medium before addition to cell cultures. Test compounds were then added to the culture medium for designated various concentrations. After 48 h, an amount of 25 μL of MTT was added to each well, and the samples were incubated at 37° C. for 4 h. A 100 μL solution of lysis buffer containing 20% SDS and 50% N,N-dimethylformamide was added to each well and incubated at 37° C. for another 16 h. The absorbency at 550 nm was measured using an ELISA reader. The results of this assay are provided in Table.

### Example 6

### Telomerase Assay

Telomeric repeat amplification protocol (TRAP) was utilized for telomerase activity assay. The telomerase products were resolved by 0.% polyacrylamide gel electrophoresis and visualized by staining with SYBER Green. As a source of telomerase, the total cell lysates derived from lung cancer cell line H1299 cells were used. Protein concentration of the lysates was assayed using Bio-Rad protein assay kit using BSA standards. The results of this assay are provided in Table.

### Example 7

### SEAP Assay

Secreted alkaine phosphatase was used as the reporter system to monitor the transcriptional activity of hTERT. Here, about 1 cells each were grown in 96-well plates and incubated at 37° C. for 24 h and changed with fresh media. Varying amounts of drugs were added and cells were incubated for another 24 h. Culture media were collected and heated at 65° C. for 10 min to inactivate heat-labile phosphatases. An equal amount of SEAP buffer (2 M diethanolamine, 1 mM MgCl₂, and 20 mM 1-homoarginine) was added to the media and p-nitrophenyl phosphate was added to a final concentration of 12 mM. Absorptions at 405 nm were taken, and the rate of absorption increase is determined. The results of this assay are provided in Table.

### Example 8

### Cell Culture and Assessment of hTERT

Non small lung cancer cells H1299 (telomerase positive) were grown in RPMI 1640 media supplemented with 10% fetal bovine serum, 100 units/mL penicillin and 100 mg/mL streptomycin in a humidified atmosphere with 5% CO₂ at 37° C. The hTERT immortalized hTERT-BJ1 (BD Biosciences Clontech)³⁹ were grown in DMEM media supplemented with 10% fetal calf serum, 100 units/mL penicillin and 100 mg/mL streptomycin, 1 mM sodium pyruvate, and 4 mM I-arginine in a humidified atmosphere with 5% CO₂ at 37° C. Culture media were changed every 3 days. To establish stable cell lines that the expression of hTERT could be monitored by a reporter system, a 3.3 kbp DNA fragment ranging from −3338 to +1 bp of the hTERT gene was subcloned upstream to a secreted alkaline phosphatase gene (SEAP) and transfectectd into H1299 or hTERT-BJ1 by electroporation. The stable clones were selected using G418. The stable clones derived from H1299 or hTERT-BJ1 were cultured using conditions that are similar to their parental cells. The results of this assay are provided in Table.

The contents of all patents, patent applications, published articles, books, reference manuals and abstracts cited herein and hereby incorporated by reference in their entirety to more fully describe the state of the art to which the invention pertains.

As various changes can be made in the above-described subject matter without departing from the scope of the invention, it is intended that all subject matter contained in the above description, shown in the accompanying drawing, or defined in the appended claims, be interpreted as descriptive, illustrative, or non-limiting. Modifications and variations of the present invention are possible in light of the above teachings. It is therefore to be understood that within the scope of the appended claims, the invention may be practiced otherwise than as specifically described.

The ratio of relative cell viability under relative SEAP activity is over 1.2. All of SEAP data are shown as the result that drug-self interference has been subtracted

TABLE 1
Cytotoxicity Against the Growth of Suspended Murine and Human Tumor
Cell Lines and Inhibitory Effect of Anthraquinone Derivatives (IIa-k)on
Iron-induced Lipid Peroxidation in Rat Brain Homogenates.

| | | IC₅₀ (μM)^{a} | | LP (%) |
|---|---|---|---|---|
| Compound | R | Hep G2^{c} | C6 cells^{d} | (10 mM)^{b} |
| IIa | CH₂CH₃ | 12.2 ± 1.1 | 0.02 ± 0.01 | 83 ± 2.2 |
| IIb | CH₂CH₂OH | 36.4 ± 1.5 | 21.5 ± 0.8 | 16 ± 2.2 |
| IIc | CH₂CH₂CH₃ | 75.1 ± 2.5 | 29.9 ± 2.1 | 15 ± 1.5 |
| IId | CH₂CH(OH)CH₂OH | 34.3 ± 1.8 | 38.5 ± 1.5 | 83 ± 1.1 |
| IIe | (CH₂)₆OH | 49.3 ± 2.1 | 31.7 ± 1.6 | 54 ± 1.9 |
| IIf | 2-NH₂C₆H₄ | 34.0 ± 1.7 | 15.1 ± 1.7 | 5 ± 0.5 |
| IIg | 3-NH₂C₆H₄ | 21.5 ± 1.2 | 26.3 ± 2.8 | 6 ± 0.9 |
| IIh | 4-NH₂C₆H₄ | 17.4 ± 1.5 | 0.05 ± 0.01 | 20 ± 1.4 |
| IIi | CH₂C₆H₅ | 41.5 ± 2.5 | 38.2 ± 4.4 | >100 |
| IIj | CH₂C₆H₄(OCH₃)(p) | 28.6 ± 1.2 | 25.1 ± 2.8 | 67 ± 2.9 |
| IIk | CH₂CH₂C₆H₅ | 36.9 ± 1.5 | 32.9 ± 3.3 | 69 ± 1.5 |
| | Mitoxantrone | 2.0 ± 0.5 | 0.07 ± 0.01 | 54 ± 1.5 |
| | Ascorbic acid | | | >100 |
| | (+)−α-Tocopherol | | | >100 |
| | Anthrarufin | | | −36 ± 1.9 |
| ^{a}IC_{50,} drug concentration inhibiting 50% of cellular growth following 48 h of drug exposure. Values are in μM and represent an average of three experiments. The variance for the IC₅₀ values was less than ±20%. Inhibition of cell growth was | | | | |
| # significantly different with respect to that of the control; n = 3 or more, P < 0.01. | | | | |

^{b}Relative percentage of inhibition. Inhibition was compared with that of the control (ascorbic acid, α-tocopherol and mitoxantrone-HCl), P < 0.01, mean ± S.E., n = 4. Values are mean percent inhibition at the indicated concentration (μM), and standard errors.
^{c}Hep G2, human hepatoma G2 cells.
^{d}C6 cells, rat glioma C6 cells.

TABLE 2
Inhibitory Effects of IIi on Iron-induced Lipid
Peroxidation in Rat Brain Homogenates.

| | Inhibition (%)^{a} | | | |
|---|---|---|---|---|
| Compound | 10 mM | 1 mM | 0.1 mM | 0.01 mM |
| IIi | >100 | 95 | 60 ± 2.0 | 24 ± 0.8 |
| Ascorbic acid | 100 | 75 ± 1.5 | 32 ± 1.2 | 10 ± 0.6 |
| (+)−α-Tocopherol | 100 | 55 ± 1.7 | 0 | 0 |
| Mitoxantrone-HCl | 100 | 54 ± 2.1 | 22 ± 3.5 | 5 ± 0.3 |

^{a}Relative percentage of inhibition. Inhibition was compared to that of the control (ascorbic acid, (+)-α-tocopherol and mitoxantrone-HCl); P < 0.01, mean ± S.E., n = 4. Values are mean percent inhibition at the indicated concentration (mM) with standard errors.

TABLE 3
Cytotoxicity against the growth of suspended murine and human tumors
and inhibitory effect of anthraquinone derivatives (IIIa-n) on
iron-induced lipid peroxidation in rat brain homogenates.

| | | IC₅₀ (μM)^{a} | | Inhibition of |
|---|---|---|---|---|
| Compd | R | Hep G2^{c} | C6 cells^{d} | LP(10 mM)^{b} |
| IIIa | CH₂CH₃ | 4.1 ± 0.5 | 21.1 ± 1.6 | −100 |
| IIIb | CH₂CH₂CH₃ | 0.02 ± 0.01 | 38.5 ± 2.8 | 54 ± 2.2 |
| IIIc | CH₂CH₂CH₂CH₂CH₃ | 36.2 ± 2.5 | 39.1 ± 4.1 | −55 ± 1.5 |
| IIId | C(CH₃)₃ | 13.7 ± 1.8 | 12.0 ± 1.5 | −23 ± 1.1 |
| IIIe | C₆H₅ | 47.7 ± 5.5 | 38.7 ± 3.6 | −50 ± 1.9 |
| IIIf | 2-ClC₆H₄ | 0.04 ± 0.01 | 40.7 ± 4.7 | 5 ± 0.5 |
| IIIg | 3-ClC₆H₄ | 15.1 ± 1.9 | 25.1 ± 2.8 | 1 ± 0.1 |
| IIIh | 4-ClC₆H₄ | 48.1 ± 4.5 | 38.6 ± 3.5 | 2 ± 0.2 |
| IIIi | 2,4-Cl₂C₆H₃ | >50 | 38.4 ± 4.4 | −1 ± 0.1 |
| IIIj | 2-CH₃C₆H₄ | 21.6 ± 2.2 | 25.1 ± 2.8 | 23 ± 1.1 |
| IIIk | 3-CH₃C₆H₄ | 18.1 ± 1.5 | 30.1 ± 3.3 | −32 ± 1.5 |
| IIIl | 4-CH₃C₆H₄ | 9.3 ± 0.9 | 37.6 ± 4.1 | 33 ± 1.2 |
| IIIm | CH₂C₆H₅ | 9.0 ± 1.5 | 39.1 ± 6.2 | −1 ± 0.2 |
| IIIn | CH₂CH₂C₆H₅ | 0.4 ± 0.1 | 40.1 ± 5.5 | >100 |
| | mitoxantrone | 2.0 ± 0.5 | 0.07 ± 0.01 | >100 |
| | ascorbic acid | | | >100 |
| | (+)−α-tocopherol | | | >100 |
| | anthrarufin | | | −36 ± 1.1 |
| ^{a}IC₅₀, drug concentration inhibiting 50% of cellular growth following 48 h of drug exposure. Values are in μM and represent an average of three experiments. The variance for the IC₅₀ was less than ±20%. | | | | |
| # Inhibition of cell growth was significantly different with respect to that of the control; N = 3 or more, P < 0.01. | | | | |
| ^{b}Relative percentage of inhibition. Inhibition was compared to that of the control [ascorbic acid, α-tocopherol and mitoxantrone-HCl], P < 0.01, Mean ± S.E., n = 4. Values are mean percent inhibition | | | | |
| # at the indicated concentration (mM), and standard errors. | | | | |

^{c}Hep G2: human hepatoma G2 cells.
^{d}C6 cells: rat glioma C6 cells.

TABLE 4
Inhibitory effects of IIIn on iron-induced lipid
peroxidation in rat brain homogenates.

| | Inhibition (%)^{a} | | | |
|---|---|---|---|---|
| Compound | 10 mM | 1 mM | 0.1 mM | 0.01 mM |
| IIIn | >100 | >100 | 95 ± 2.0 | 50 ± 0.8 |
| ascorbic acid | 100 | 75 ± 1.5 | 32 ± 1.2 | 10 ± 0.6 |
| (+)−α-tocopherol | 100 | 55 ± 1.7 | 0 | 0 |
| mitoxantrone-HCl | 100 | 54 ± 2.1 | 22 ± 3.5 | 5 ± 0.3 |
| ^{a}Relative percentage of inhibition. Inhibition was compared to that of the control ascorbic acid, (+)−α-tocopherol and mitoxantrone-HCl], P < 0.01, Mean ± S.E., n = 4. Values are mean percent inhibition | | | | |
| # at the indicated concentration (mM), and standard errors. | | | | |

TABLE 5
In vitro Cytotoxicity of Diaminoanthraquinones (IVa-s) Against
the Growth of Suspended Murine and Human Tumor Cell Lines

| | | IC₅₀ (μM)^{a} | | |
|---|---|---|---|---|
| Compound | R | Hep G2^{b} | C6 cells^{c} | 2.2.15^{d} |
| IVa | CH₂CH₃ | >20 | >20 | >20 |
| IVb | CH₂CH₂OH | >20 | >20 | 19.26 ± 2.2 |
| IVc | CH(CH₃)₂ | 7.64 ± 2.38 | >20 | 18.98 ± 2.4 |
| IVd | CH₂CH₂N(CH₃)₂ | 0.09 ± 0.01 | 0.12 ± 0.01 | 0.13 ± 0.01 |
| IVe | CH₂CH₂NH(CH₂)₂OH | 1.20 ± 0.02 | 1.17 ± 0.03 | 8.35 ± 0.11 |
| IVf | CH₂CH₂CH₃ | 1.74 ± 0.14 | 16.03 ± 0.68 | 1.94 ± 0.02 |
| IVg | CH₂CH(CH₃)₂ | 14.27 ± 1.54 | 12.67 ± 0.37 | >20 |
| IVh | CH₂CH₂CH₂OH | >20 | >20 | >20 |
| IVi | CH₂CH₂CH₂NH₂ | 11.67 ± 0.09 | 12.18 ± 0.04 | 7.48 ± 0.09 |
| IVj | CH₂CH₂CH₂CH₃ | >20 | >20 | >20 |
| IVk | CH₂CH₂CH₂CH₂OH | >20 | >20 | 10.30 ± 0.24 |
| IVl | CH₂CH₂CH₂CH₂NH₂ | 11.61 ± 0.02 | 12.56 ± 0.16 | 10.07 ± 0.58 |
| IVm | CH₂CH₂CH₂CH₂CH₂NH₂ | 17.37 ± 0.74 | 8.36 ± 0.13 | >20 |
| IVn | CH₂CH₂CH₂CH₂CH₂CH₃ | >20 | >20 | >20 |
| IVo | cyclopentane | >20 | >20 | >20 |
| IVp | 2,3-(CH₃)₂-cyclohexane | >20 | >20 | >20 |
| IVq | 4-OHC₆H₄ | 4.20 ± 0.76 | 14.21 ± 0.08 | >20 |
| IVr | CH₂C₆H₅ | >20 | >20 | >20 |
| IVs | CH₂CH₂C₆H₅ | >20 | >20 | >20 |
| | Mitoxantrone | 2.00 ± 0.5 | 0.07 ± 0.01 | 0.40 ± 0.02 |
| | Adriamycin | 0.90 ± 0.01 | 1.00 ± 0.16 | 1.60 ± 0.04 |
| | Cisplatin | 1.48 ± 0.62 | >1 | 2.0 ± 0.54 |
| ^{a}IC₅₀, drug concentration inhibiting 50% of cellular growth following 48 h of drug exposure. Values are in μM and represent an average of three experiments. The variance for the IC₅₀ values was less than ±20%. | | | | |
| # Inhibition of cell growth was significantly different with respect to that of the control; n = 3 or more, P < 0.01. Inhibition was compared with that of control (mitoxantrone-HCl, adriamycin, cisplatin), (μM), and standard errors. | | | | |

^{b}Hep G2, human hepatoma G2 cells.
^{c}C6 cells, rat glioma C6 cells.
^{d}2.2.15 cells, hepatitis B virus transfected hepatoma cell lines, HepG 2.2.15 cells.

TABLE 6
Effects of Symmetrical 1,5-Diaminoanthraquinones
(IVa-p) on Activating hTERT Expression

| | | P_{hTERT}-SEAP (hTERT-BJ1)^{b} | | | |
|---|---|---|---|---|---|
| | | Conc. | Relative MTT | Relative SEAP | SEAP/ |
| No. | R | (μM)^{a} | viability (%) | activity (%) | vibility |
| IVa | CH₂CH₃ | 3.3 | 100 ± 4.8 | 94 ± 17.4 | 0.94 |
| | | 33 | 97 ± 1.0 | 96 ± 8.5 | 1.00 |
| | | 339 | 99 ± 3.2 | 66 ± 11.8 | 0.67 |
| IVb | CH₂CH₂OH | 3.0 | 116 ± 5.6 | 45 ± 20.6 | 0.39 |
| | | 30 | 90 ± 26.9 | 16 ± 13.1 | 0.18 |
| | | 308 | 69 ± 18.7 | 11 ± 11.6 | 0.16 |
| IVc | CH(CH₃)₂ | 3.1 | 101 ± 4.5 | 32 ± 18.3 | 0.32 |
| | | 31 | 93 ± 6.5 | 23 ± 20.5 | 0.25 |
| | | 310 | 98 ± 12.9 | (−2) ± 15.3 | −0.02 |
| IVd | CH₂CH₂N(CH₃)₂ | 2.6 | 92 ± 4.8 | 11 ± 22.4 | 0.12 |
| | | 26 | 76 ± 5.9 | (−15) ± 18.2 | −0.19 |
| | | 262 | 7 ± 18.2 | (−26) ± 16.9 | −3.77 |
| IVe | CH₂CH₂NH(CH₂)₂OH | 2.4 | 84 ± 19.8 | 60 ± 11.6 | 0.72 |
| | | 24 | 60 ± 11.2 | 40 ± 17.3 | 0.67 |
| | | 242 | 44 ± 12.9 | 52 ± 19.1 | 1.18 |
| IVf | CH₂CH₂CH₃ | 3.1 | 97 ± 5.6 | 18 ± 16.7 | 0.18 |
| | | 31 | 93 ± 8.9 | 19 ± 24.6 | 0.20 |
| | | 310 | 42 ± 8.1 | (−7) ± 27.1 | −0.16 |
| IVg | CH₂CH(CH₃)₂ | 2.8 | 110 ± 7.6 | 22 ± 18.3 | 0.20 |
| | | 28 | 103 ± 3.0 | 22 ± 21.4 | 0.21 |
| | | 285 | 72 ± 3.3 | 29 ± 3.9 | 0.41 |
| IVh | CH₂CH₂CH₂OH | 2.8 | 72 ± 7.4 | 41 ± 12.5 | 0.57 |
| | | 28 | 39 ± 10.5 | 0 ± 22.1 | 0.01 |
| | | 282 | 26 ± 15.9 | (−3) ± 10.0 | −0.10 |
| IVi | CH₂CH₂CH₂NH₂ | 2.8 | 85 ± 6.9 | 103 ± 19.8 | 1.22 |
| | | 28 | 3 ± 6.9 | 47 ± 20.5 | 15.35 |
| | | 283 | (−2) ± 7.7 | 60 ± 15.1 | −28.94 |
| IVj | CH₂CH₂CH₂CH₃ | 2.8 | 109 ± 9.1 | 98 ± 27.4 | 0.89 |
| | | 28 | 99 ± 5.9 | 103 ± 27.0 | 1.04 |
| | | 285 | 75 ± 5.9 | 123 ± 22.9 | 1.64 |
| IVk | CH₂CH₂CH₂CH₂NH₂ | 2.6 | 101 ± 10.5 | 114 ± 20.5 | 1.13 |
| | | 26 | 101 ± 8.8 | 113 ± 21.6 | 1.12 |
| | | 265 | 91 ± 11.8 | 127 ± 19.9 | 1.39 |
| IVl | CH₂CH₂CH₂CH₂CH₂CH₃ | 2.4 | 106 ± 3.9 | 90 ± 19.5 | 0.85 |
| | | 24 | 97 ± 4.6 | 97 ± 17.9 | 1.00 |
| | | 245 | 68 ± 8.5 | 122 ± 30.2 | 1.79 |
| IVm | cyclopentane | 2.6 | 104 ± 3.6 | 18 ± 20.1 | 0.18 |
| | | 26 | 102 ± 4.0 | 14 ± 29.6 | 0.14 |
| | | 267 | 87 ± 4.6 | 34 ± 16.5 | 0.39 |
| IVn | 2,3-(CH₃)₂-cyclohexane | 2.1 | 118 ± 10.6 | 94 ± 20.2 | 0.80 |
| | | 21 | 99 ± 8.2 | 92 ± 17.4 | 0.93 |
| | | 218 | 84 ± 6.9 | 90 ± 22.7 | 1.07 |
| IVo | CH₂C₆H₅ | 2.3 | 110 ± 4.1 | 110 ± 14.8 | 1.00 |
| | | 23 | 102 ± 5.3 | 92 ± 13.8 | 0.91 |
| | | 238 | 74 ± 4.9 | 86 ± 13.8 | 1.15 |
| IVp | CH₂CH₂C₆H₅ | 2.2 | 97 ± 4.0 | 115 ± 12.9 | 1.18 |
| | | 22 | 92 ± 1.0 | 103 ± 12.3 | 1.12 |
| | | 223 | 81 ± 4.7 | 133 ± 23.8 | 1.65 |
| | Mitoxnatrone | 1.9 | 75 ± 2.9 | 30 ± 5.8 | 0.40 |
| | | 19 | 56 ± 3.1 | 13 ± 9.2 | 0.24 |
| | | 193 | 10 ± 2.0 | 4 ± 14.2 | 0.38 |
| ^{a}Values are in μM and represent an average of three experiments. The variance for the relative viability (%) and relative SEAP activity (%) values was less than ±20% . Activity of P_{hTERT}-SEAP (hTERT-BJ1) cell growth was | | | | | |
| # significantly different from that of the control; n = 3 or more, P < 0.05. Relative percentage of inhibition was not compared with that of the control, P < 0.01, mean ± S.E., n = 4. Values are mean percent activity at the indicated concentration, and standard errors. | | | | | |

^{b}The hTERT immortalized hTERT-BJ1 was purchased from BD Biosciences Clontech.
Note:
The results in this column are shown as means ± SE of experiments repeated five times. The different symbols qualify as in any concentration of treatment: Relative Cell Viability >80%, Relative SEAP activity >100% and P value below 0.05 analyzed with Two-tail T-test.

TABLE 7
Effects of Symmetrical 1,5-Diaminoanthraquinones
(IVa-p) on Repressing hTERT Expression

| | | P_{hTERT}-SEAP (hTERT-H1299)^{b} | | | |
|---|---|---|---|---|---|
| | | Conc | Relative MTT | Relative SEAP | SEAP/ |
| No. | R | (μM)^{a} | viability (%) | activity (%) | vibility |
| IVa | CH₂CH₃ | 3.3 | 106 ± 7.4 | 108 ± 6.3 | 1.01 |
| | | 33 | 104 ± 3.7 | 101 ± 6.8 | 0.97 |
| | | 339 | 103 ± 6.4 | 95 ± 11.0 | 0.91 |
| IVb | CH₂CH₂OH | 3.0 | 105 ± 6.3 | 91 ± 3.9 | 0.86 |
| | | 30 | 88 ± 9.1 | 88 ± 4.5 | 1.00 |
| | | 308 | 53 ± 2.8 | 5.7 ± 1.1 | 1.08 |
| IVc | CH(CH₃)₂ | 3.1 | 114 ± 7.6 | 94 ± 4.1 | 0.82 |
| | | 31 | 111 ± 4.7 | 93 ± 2.0 | 0.84 |
| | | 310 | 50 ± 7.5 | 66 ± 4.7 | 1.32 |
| IVd | CH₂CH₂N(CH₃)₂ | 2.6 | 107 ± 9.1 | 103 ± 6.0 | 0.95 |
| | | 26 | 81 ± 8.3 | 54 ± 5.9 | 0.67 |
| | | 262 | 29 ± 2.6 | 40 ± 5.9 | 1.39 |
| IVe | CH₂CH₂NH(CH₂)₂OH | 2.4 | 97 ± 8.7 | 87 ± 3.8 | 0.89 |
| | | 24 | 37 ± 3.9 | 43 ± 5.5 | 1.15 |
| | | 242 | 11 ± 4.1 | 40 ± 6.7 | 3.77 |
| IVf | CH₂CH₂CH₃ | 3.1 | 99 ± 7.6 | 101 ± 15.3 | 1.02 |
| | | 31 | 98 ± 5.8 | 100 ± 5.6 | 1.03 |
| | | 310 | 46 ± 9.5 | 89 ± 8.3 | 1.93 |
| IVg | CH₂CH(CH₃)₂ | 2.8 | 108 ± 4.8 | 94 ± 7.0 | 0.87 |
| | | 28 | 106 ± 3.7 | 91 ± 2.5 | 0.85 |
| | | 285 | 80 ± 4.8 | 75 ± 4.1 | 0.94 |
| IVh | CH₂CH₂CH₂OH | 2.8 | 107 ± 5.0 | 88 ± 4.9 | 0.82 |
| | | 28 | 49 ± 3.5 | 69 ± 5.4 | 1.41 |
| | | 282 | 40 ± 10.4 | 33 ± 2.2 | 0.81 |
| IVi | CH₂CH₂CH₂NH₂ | 2.8 | 85 ± 11.5 | 102 ± 4.9 | 1.20 |
| | | 28 | 32 ± 9.3 | 44 ± 7.5 | 1.39 |
| | | 283 | 16 ± 2.1 | 38 ± 6.0 | 2.34 |
| IVj | CH₂CH₂CH₂CH₃ | 2.8 | 102 ± 7.0 | 106 ± 7.1 | 1.04 |
| | | 28 | 104 ± 9.2 | 105 ± 5.5 | 1.01 |
| | | 285 | 81 ± 13.9 | 98 ± 5.2 | 1.21 |
| IVk | CH₂CH₂CH₂CH₂NH₂ | 2.6 | 114 ± 8.1 | 89 ± 4.9 | 0.78 |
| | | 26 | 110 ± 7.1 | 71 ± 9.8 | 0.65 |
| | | 265 | 16 ± 3.4 | 27 ± 2.0 | 1.65 |
| IVl | CH₂CH₂CH₂CH₂CH₂CH₃ | 2.4 | 99 ± 5.3 | 101 ± 6.1 | 1.02 |
| | | 24 | 95 ± 6.2 | 106 ± 9.1 | 1.11 |
| | | 245 | 85 ± 8.8 | 104 ± 9.4 | 1.22 |
| IVm | cyclopentane | 2.6 | 114 ± 7.5 | 98 ± 4.5 | 0.86 |
| | | 26 | 110 ± 5.7 | 95 ± 2.8 | 0.86 |
| | | 267 | 86 ± 8.2 | 90 ± 4.8 | 1.05 |
| IVn | 2,3-(CH₃)₂-cyclohexane | 2.1 | 88 ± 7.9 | 103 ± 9.2 | 1.17 |
| | | 21 | 84 ± 10.2 | 105 ± 5.1 | 1.25 |
| | | 218 | 58 ± 8.0 | 96 ± 4.5 | 1.64 |
| IVo | CH₂C₆H₅ | 2.3 | 98 ± 8.4 | 99 ± 5.0 | 1.01 |
| | | 23 | 100 ± 7.3 | 103 ± 8.9 | 1.04 |
| | | 238 | 40 ± 8.4 | 97 ± 3.8 | 2.45 |
| IVp | CH₂CH₂C₆H₅ | 2.2 | 94 ± 5.2 | 105 ± 3.0 | 1.12 |
| | | 22 | 97 ± 3.2 | 103 ± 9.5 | 1.06 |
| | | 223 | 77 ± 4.2 | 96 ± 2.9 | 1.25 |
| | Mitoxnatrone | 1.9 | 100 ± 5.6 | 81 ± 3.8 | 0.82 |
| | | 19 | 57 ± 4.3 | 66 ± 4.0 | 1.16 |
| | | 193 | 39 ± 3.2 | 47 ± 3.9 | 1.21 |
| ^{a}Values are in μM and represent an average of three experiments. The variance for the relative viability (%) and relative SEAP activity (%) values was less than ±20%. Repression of P_{hTERT}-SEAP (hTERT-H1299) cell growth was significantly different | | | | | |
| # from that of the control; n = 3 or more, P < 0.05. Relative percentage of inhibition was not compared with that of the control, P < 0.01, mean ± S.E., n = 4. Values are mean percent activity at the indicated concentration, and standard errors. | | | | | |

^{b}The hTERT cancer cell hTERT-H1299 was purchased from BD Biosciences Clontech.

TABLE 8
In vitro Cytotoxicity of 1,8-Diaminoanthraquinones (Va-p) Against
the Growth of Suspended Murine and Human Tumor Cell Lines

| | | IC₅₀(μM)^{a} | | |
|---|---|---|---|---|
| Compound | R | C6 cells^{c} | Hep G2^{b} | 2.2.15^{d} |
| Va | CH₂CH₃ | >20 | >20 | >20 |
| Vb | CH₂CH₂CH₃ | 0.61 ± 0.01 | 0.19 ± 0.01 | 1.06 ± 0.03 |
| Vc | CH₂CH₂CH₂CH₃ | >20 | >20 | >20 |
| Vd | CH₂CH(CH₃)₂ | 1.32 ± 0.01 | >20 | >20 |
| Ve | (CH₂)₅CH₃ | >20 | >20 | >20 |
| Vf | CH(CH₃)₂ | 1.24 ± 0.01 | >20 | >20 |
| Vg | CH₂CH₂OH | 0.02 ± 0.01 | >20 | >20 |
| Vh | CH₂CH₂CH₂OH | 1.00 ± 0.01 | >20 | >20 |
| Vi | CH₂CH₂CH₂CH₂OH | 0.41 ± 0.02 | 1.65 ± 0.13 | >20 |
| Vj | CH₂CH₂N(CH₃)₂ | 0.15 ± 0.04 | 0.16 ± 0.04 | 8.55 ± 0.09 |
| Vk | CH₂CH₂CH₂NH₂ | >20 | 11.43 ± 0.17 | >20 |
| Vl | CH₂CH₂CH₂CH₂NH₂ | >20 | 11.47 ± 0.34 | >20 |
| Vm | CH₂CH₂CH₂CH₂CH₂NH₂ | 0.11 ± 0.01 | 0.09 ± 0.01 | 1.29 ± 0.06 |
| Vn | Cyclopentane | >20 | >20 | >20 |
| Vo | CH₂C₆H₅ | 1.66 ± 0.09 | >20 | >20 |
| Vp | CH₂CH₂C₆H₅ | >20 | >20 | >20 |
| | Mitoxantrone | 0.07 ± 0.01 | 2.0 ± 0.50 | 0.40 ± 0.02 |
| | Adriamycin | 1.00 ± 0.16 | 0.90 ± 0.01 | 1.60 ± 0.04 |
| ^{a}IC₅₀, drug concentration inhibiting 50% of cellular growth following 48 h of drug exposure. Values are in μM and represent an average of three experiments. The variance for the IC₅₀ values was less than ±20%. Inhibition of cell growth was | | | | |
| # significantly different with respect to that of the control; n = 3 or more, P < 0.01. Inhibition was compared with that of the control (mitoxantrone-HCl, adriamycin, cisplatin), (μM), and standard errors. | | | | |

^{b}Hep G2, human hepatoma G2 cells.
^{c}C6 cells, rat glioma C6 cells.
^{d}2.2.15 cells, hepatitis B virus transfected hepatoma cell lines, HepG 2.2.15 cells.

TABLE 9
In vitro Cytotoxicity of 1,4-Diamidoanthraquinones (VI₁₋₃₇) Against
the Growth of Suspended Murine and Human Tumor Cell Lines

| | | IC₅₀ (μM)^{a} | | |
|---|---|---|---|---|
| Compound | R | C6 cells^{c} | Hep G2^{b} | 2.2.15^{d} |
| VI₁ | CH₂Cl | >20 | >20 | >20 |
| VI₂ | 2-ClC₆H₄ | >20 | >20 | >20 |
| VI₃ | CH₃ | >20 | >20 | >20 |
| VI₄ | C₆H₅ | >20 | >20 | >20 |
| VI₅ | 3-ClC₆H₄ | >20 | >20 | >20 |
| VI₆ | 3-CH₃C₆H₄ | >20 | >20 | >20 |
| VI₇ | CH₂CH₂Cl | >20 | >20 | >20 |
| VI₈ | 2,4-Cl₂C₆H₃ | >20 | >20 | >20 |
| VI₉ | CHClCH₃ | >20 | >20 | >20 |
| VI₁₀ | 2-FC₆H₄ | >20 | >20 | >20 |
| VI₁₁ | 2-NO₂C₆H₄ | >20 | >20 | >20 |
| VI₁₂ | 3-FC₆H₄ | >20 | >20 | >20 |
| VI₁₃ | 2,4,6-Cl₃C₆H₂ | >20 | 2.77 ± 0.93 | 3.63 ± 2.33 |
| VI₁₄ | 2,3,6-F₃C₆H₂ | >20 | >20 | >20 |
| VI₁₅ | 2,4,5-Cl₃C₆H₂ | >20 | >20 | 3.35 ± 3.24 |
| VI₁₆ | 4-ClC₆H₄ | >20 | >20 | >20 |
| VI₁₇ | cyclohexane | >20 | >20 | >20 |
| VI₁₈ | 2,4-F₂C₆H₃ | >20 | >20 | >20 |
| VI₁₉ | (CH₂)₂CH(CH₂)₄ | >20 | >20 | >20 |
| VI₂₀ | cyclopentane | >20 | >20 | >20 |
| VI₂₁ | cyclopropane | >20 | >20 | >20 |
| VI₂₂ | 2-SC(CH)₃ | >20 | >20 | >20 |
| VI₂₃ | 2,3-Cl₂-5-FC₆H₂ | >20 | >20 | >20 |
| VI₂₄ | 2-OC(CH)₃ | >20 | >20 | >20 |
| VI₂₅ | CH₂-2-S—C(CH)₃ | >20 | >20 | 15.47 ± 12.00 |
| VI₂₆ | 3-O-2,5-(CH₃)₂CH | >20 | >20 | >20 |
| VI₂₇ | CH(CH₂)CHC₆H₅ | >20 | >20 | 19.48 ± 18.13 |
| VI₂₈ | CH₂SC₆H₅ | >20 | >20 | >20 |
| VI₂₉ | C₆H₃(CF₃)₂(o,m) | 2.82 ± 0.47 | >20 | 2.20 ± 0.64 |
| VI₃₀ | C₆H₄F(p) | >20 | >20 | >20 |
| VI₃₁ | C₆H₄CF₃(p) | >20 | >20 | >20 |
| VI₃₂ | CH₂C₆H₄F(p) | >20 | >20 | >20 |
| VI₃₃ | CH₂N(CH₂CH₃)₂ | 4.46 ± 2.76 | 0.65 ± 0.62 | 12.97 ± 11.93 |
| VI₃₄ | (CH₂)₂N(CH₂CH₃)₂ | 0.90 ± 0.68 | 0.49 ± 0.41 | 0.28 ± 0.01 |
| VI₃₅ | CHCH₃N(CH₂CH₃)₂ | 18.09 ± 14.11 | 2.02 ± 0.26 | 8.57 ± 7.18 |
| VI₃₆ | CHCH₃NHCH₂CH(CH₂)₂ | 2.76 ± 1.74 | 6.46 ± 1.40 | 3.46 ± 1.15 |
| VI₃₇ | CH₂CH₂NHCH₂CH(CH₂)₂ | 0.40 ± 0.09 | 0.32 ± 0.29 | 1.71 ± 1.67 |
| | Mitoxantrone | 0.07 ± 0.01 | 2.0 ± 0.50 | 0.40 ± 0.02 |
| | Adriamycin | 1.00 ± 0.16 | 0.90 ± 0.01 | 1.60 ± 0.04 |
| ^{a}IC₅₀, drug concentration inhibiting 50% of cellular growth following 48 h of drug exposure. Values are in μM and represent an average of three experiments. The variance for the IC₅₀ values was less than ±20%. Inhibition of cell growth was | | | | |
| # significantly different with respect to that of the control; n = 3 or more, P < 0.01. Inhibition was compared with that of the control (mitoxantrone-HCl, adriamycin, cisplatin), (μM), and standard errors. | | | | |

^{b}Hep G2, human hepatoma G2 cells.
^{c}C6 cells, rat glioma C6 cells.
^{d}2.2.15 cells, hepatitis B virus transfected hepatoma cell lines, HepG 2.2.15 cells.

TABLE 10
Effects of Symmetrical 1,5-bis-thio-Substituted Anthraquinones
(IIa-o) on Respressing and Activating hTERT Expression

| | | | P_{hTERT}-SEAP (H1299)^{b} | | P_{hTERT}-SEAP (hTERT-BJ1)^{c} | |
|---|---|---|---|---|---|---|
| | | Conc. | Relative | Relative SEAP | Relative | Relative SEAP |
| No. | R | (μM)^{a} | viability (%) | activity (%) | viability (%) | activity (%) |
| IIa | CH₂CH₃ | 3.0 | 111 ± 2.8 | 134 ± 14.4 | 112 ± 9.2 | 109 ± 22.4 |
| | | 30 | 44 ± 7.3 | 111 ± 7.7 | 98 ± 12.3 | 110 ± 14.3 |
| | | 300 | 25 ± 2.3 | 99 ± 14.3 | 34 ± 18.1 | 104 ± 20.6 |
| IIb | CH₂CH₂OH | 2.8 | 54 ± 4.0 | 97 ± 15.7 | 94 ± 9.3 | 104 ± 15.7 |
| | | 28 | 29 ± 7.0 | 76 ± 12.4 | 49 ± 2.9 | 98 ± 10.9 |
| | | 280 | 36 ± 7.2 | 45 ± 2.7 | 23 ± 2.9 | 71 ± 5.0 |
| IIc | CH₂CH₂CH₃ | 2.8 | 96 ± 5.9 | 73 ± 5.4 | 103 ± 6.3 | 132 ± 21.0 |
| | | 28 | 44 ± 2.5 | 29 ± 2.5 | 97 ± 3.0 | 110 ± 13.2 |
| | | 280 | 25 ± 2.4 | 17 ± 13.9 | 39 ± 4.9 | 122 ± 14.3 |
| IId | CH₂CH(OH)CH₂OH | 2.4 | 102 ± 6.2 | 105 ± 21.6 | 98 ± 10.7 | 144 ± 16.9 |
| | | 24 | 103 ± 4.2 | 90 ± 5.7 | 86 ± 5.5 | 136 ± 10.0 |
| | | 240 | 83 ± 18.2 | 81 ± 6.1 | 79 ± 8.2 | 142 ± 9.1 |
| IIe | (CH₂)₆OH | 2.1 | 99 ± 6.2 | 110 ± 6.1 | 99 ± 6.2 | 140 ± 9.7 |
| | | 21 | 94 ± 3.9 | 100 ± 5.5 | 70 ± 2.2 | 128 ± 14.4 |
| | | 210 | 36 ± 4.2 | 68 ± 5.9 | 40 ± 4.7 | 75 ± 17.4 |
| IIf | 2-NH₂C₆H₄ | 2.2 | 94 ± 3.5 | 108 ± 12.2 | 103 ± 7.9 | 136 ± 17.5 |
| | | 22 | 50 ± 3.3 | 96 ± 8.4 | 107 ± 5.5 | 141 ± 18.2 |
| | | 220 | 14 ± 2.5 | 59 ± 6.6 | 25 ± 3.3 | 115 ± 29.7 |
| IIg | 3-NH₂C₆H₄ | 2.2 | 92 ± 3.5 | 106 ± 7.6 | 104 ± 5.1 | 118 ± 9.9 |
| | | 22 | 68 ± 1.9 | 109 ± 11.7 | 9 ± 3.4 | 41 ± 9.5 |
| | | 220 | 32 ± 4.9 | 101 ± 7.3 | 4 ± 1.5 | 8 ± 30.6 |
| IIh | 4-NH₂C₆H₄ | 2.2 | 103 ± 4.3 | 100 ± 5.7 | 86 ± 11.9 | 97 ± 17.9 |
| | | 22 | 76 ± 4.0 | 95 ± 2.6 | 65 ± 12.6 | 97 ± 14.5 |
| | | 220 | 42 ± 2.3 | 84 ± 5.3 | 56 ± 13.8 | 26 ± 12.6 |
| IIi | CH₂C₆H₅ | 2.2 | 83 ± 5.5 | 97 ± 6.0 | 121 ± 4.7 | 117 ± 11.3 |
| | | 22 | 44 ± 0.9 | 100 ± 7.2 | 98 ± 4.2 | 112 ± 9.3 |
| | | 220 | 34 ± 3.3 | 100 ± 12.9 | 47 ± 9.1 | 87 ± 11.1 |
| IIj | CH₂C₆H₄(OCH₃)(p) | 2.2 | 89 ± 6.1 | 92 ± 3.3 | 119 ± 9.4 | 142 ± 27.7 |
| | | 22 | 59 ± 5.4 | 96 ± 8.5 | 98 ± 13.4 | 141 ± 22.6 |
| | | 220 | 42 ± .3 | 88 ± 5.6 | 62 ± 6.4 | 118 ± 19.2 |
| IIk | CH₂CH₂C₆H₅ | 2.2 | 93 ± 6.2 | 108 ± 0.5 | 91 ± 3.9 | 119 ± 12.2 |
| | | 22 | 51 ± 9.3 | 102 ± 0.5 | 54 ± 4.4 | 109 ± 23.4 |
| | | 220 | 27 ± 2.9 | 97 ± 4.8 | 35 ± 4.1 | 110 ± 30.6 |
| IIL | C₄H₃N₂ | 2.3 | 107 ± 5.1 | 105 ± 6.6 | 104 ± 8.9 | 137 ± 12.3 |
| | | 23 | 99 ± 5.8 | 110 ± 8.0 | 103 ± 6.4 | 125 ± 6.9 |
| | | 230 | 44 ± 6.3 | 49 ± 10.0 | 73 ± 8.3 | 61 ± 8.7 |
| IIm | C₅H₄N | 2.3 | 79 ± 12.2 | 104 ± 12.6 | 112 ± 6.4 | 98 ± 12.3 |
| | | 23 | 45 ± 7.1 | 103 ± 15.8 | 91 ± 7.3 | 133 ± 6.8 |
| | | 230 | 29 ± 1.5 | 89 ± 9.3 | 55 ± 11.3 | 121 ± 13.8 |
| IIn | C₄H₂N₂(OH)(m) | 2.2 | 99 ± 4.0 | 92 ± 7.1 | 104 ± 7.6 | 93 ± 7.9 |
| | | 22 | 95 ± 4.9 | 97 ± 2.9 | 106 ± 6.3 | 116 ± 15.4 |
| | | 220 | 51 ± 16.7 | 93 ± 12.1 | 89 ± 3.5 | 145 ± 20.7 |
| IIo | C₆H₄CH₃ | 2.2 | 84 ± 5.9 | 117 ± 12.5 | 111 ± 5.4 | 132 ± 19.3 |
| | | 22 | 41 ± 2.4 | 103 ± 10.5 | 77 ± 6.9 | 109 ± 5.7 |
| | | 220 | 25 ± 2.4 | 90 ± 11.6 | 22 ± 8.5 | 87 ± 29.6 |
| ^{a}Values are in μM and represent an average of three experiments. The variance for the relative viability (%) and relative SEAP activity (%) values was less than ±20%. Activity of | | | | | | |
| P_{hTERT}-SEAP (H1299) and (hTERT-BJ1) cell growth was significantly different with respect to that of the control; n = 3 or more, P < 0.01. Relative percentage of | | | | | | |
| # inhibition was not compared with that of the control, P < 0.01, mean ± S.E., n = 4. | | | | | | |
| # Values are the mean percent activity at the indicated concentration, and include standard errors. | | | | | | |

^{b}Non-small-cell lung cancer cells H1299.
^{c}The hTERT immortalized hTERT-BJ1 was purchased from BD Biosciences Clontech.

TABLE 11
Effects of Symmetrical 1,5-Bisacyloxyanthraquinones (IIIa-n)
on Respressing and Activating hTERT Expression

| | | | P_{hTERT}-SEAP (H1299)^{b} | | P_{hTERT}-SEAP (hTERT-BJ1)^{c} | |
|---|---|---|---|---|---|---|
| | | Conc. | Relative | Relative SEAP | Relative | Relative SEAP |
| No. | R | μM^{a} | viability (%) | activity (%) | viability (%) | activity (%) |
| IIIa | COCH₂CH₃ | 2.8 | 116 ± 8.0 | 103 ± 5.8 | 99 ± 11.9 | 131 ± 7.0 |
| | | 28 | 116 ± 7.4 | 107 ± 4.9 | 109 ± 8.7 | 152 ± 16.7 |
| | | 280 | 87 ± 6.6 | 97 ± 3.1 | 93 ± 10.8 | 161 ± 12.2 |
| IIIb | COCH₂CH₂CH₃ | 2.6 | 107 ± 5.6 | 114 ± 7.9 | 109 ± 5.1 | 107 ± 11.9 |
| | | 26 | 106 ± 3.2 | 118 ± 8.4 | 106 ± 5.6 | 119 ± 8.3 |
| | | 260 | 87 ± 6.5 | 111 ± 4.4 | 85 ± 6.6 | 134 ± 3.8 |
| IIIc | CO(CH₂)₄CH₃ | 2.3 | 117 ± 5.1 | 91 ± 9.9 | 119 ± 9.4 | 147 ± 18.8 |
| | | 23 | 114 ± 4.7 | 95 ± 17.4 | 98 ± 13.4 | 149 ± 16.1 |
| | | 230 | 106 ± 4.3 | 95 ± 10.5 | 62 ± 6.4 | 124 ± 5.3 |
| IIId | COC(CH₃)₃ | 2.4 | 106 ± 8.9 | 105 ± 4.9 | 94 ± 3.4 | 147 ± 11.1 |
| | | 24 | 97 ± 7.1 | 78 ± 14.8 | 93 ± 6.0 | 165 ± 18.7 |
| | | 240 | 70 ± 5.2 | 80 ± 10.1 | 100 ± 9.0 | 141 ± 14.4 |
| IIIe | COC₆H₅ | 2.2 | 99 ± 7.3 | 88 ± 10.0 | 103 ± 6.5 | 137 ± 10.0 |
| | | 22 | 60 ± 11.9 | 94 ± 8.0 | 87 ± 9.8 | 122 ± 9.6 |
| | | 220 | 33 ± 4.7 | 86 ± 4.7 | 51 ± 4.6 | 84 ± 21.7 |
| IIIf | COC₆H₄Cl(o) | 1.9 | 74 ± 5.3 | 93 ± 4.4 | 107 ± 5.5 | 120 ± 10.5 |
| | | 19 | 34 ± 3.4 | 101 ± 4.9 | 92 ± 4.3 | 116 ± 3.7 |
| | | 190 | 30 ± 1.4 | 97 ± 5.3 | 40 ± 4.0 | 97 ± 10.8 |
| IIIg | COC₆H₄Cl(m) | 1.9 | 98 ± 3.9 | 83 ± 5.7 | 101 ± 2.3 | 154 ± 23.0 |
| | | 19 | 88 ± 7.2 | 92 ± 8.1 | 80 ± 6.7 | 152 ± 15.8 |
| | | 190 | 46 ± 3.5 | 81 ± 2.5 | 44 ± 6.0 | 91 ± 25.2 |
| IIIh | COC₆H₄Cl(p) | 1.9 | 91 ± 10.0 | 106 ± 4.5 | 111 ± 6.5 | 136 ± 5.7 |
| | | 19 | 57 ± 1.8 | 106 ± 4.9 | 89 ± 14.3 | 123 ± 6.2 |
| | | 190 | 31 ± 1.0 | 97 ± 4.9 | 48 ± 9.0 | 107 ± 6.0 |
| IIIi | COC₆H₄Cl₂(o,p) | 1.8 | 108 ± 3.8 | 100 ± 5.2 | 107 ± 7.2 | 155 ± 16.3 |
| | | 18 | 103 ± 5.9 | 102 ± 5.4 | 102 ± 4.1 | 150 ± 13.9 |
| | | 180 | 77 ± 2.8 | 96 ± 4.4 | 96 ± 7.0 | 160 ± 36.7 |
| IIIj | COC₆H₄CH₃(o) | 2.1 | 72 ± 4.9 | 97 ± 4.4 | 118 ± 11.6 | 129 ± 13.0 |
| | | 21 | 36 ± 9.7 | 91 ± 7.4 | 82 ± 8.5 | 120 ± 24.0 |
| | | 210 | 45 ± 8.0 | 90 ± 4.2 | 39 ± 13.0 | 94 ± 27.1 |
| IIIk | COC₆H₄CH₃(m) | 2.1 | 26 ± 3.7 | 104 ± 5.4 | 98 ± 9.4 | 141 ± 10.5 |
| | | 21 | 28 ± 5.0 | 116 ± 12.8 | 54 ± 7.5 | 124 ± 15.6 |
| | | 210 | 29 ± 3.3 | 110 ± 16.4 | 47 ± 4.8 | 86 ± 17.2 |
| IIIL | COC₆H₄CH₃(p) | 2.1 | 61 ± 5.8 | 98 ± 1.1 | 102 ± 13.9 | 130 ± 8.8 |
| | | 21 | 33 ± 2.8 | 95 ± 4.3 | 98 ± 6.3 | 126 ± 13.3 |
| | | 210 | 32 ± 5.7 | 95 ± 8.9 | 56 ± 6.2 | 99 ± 15.8 |
| IIIm | COCH₂C₆H₅ | 2.1 | 91 ± 4.2 | 98 ± 0.3 | 106 ± 9.0 | 129 ± 4.5 |
| | | 21 | 53 ± 2.3 | 101 ± 6.8 | 97 ± 8.2 | 125 ± 4.3 |
| | | 210 | 30 ± 1.6 | 142 ± 14 | 64 ± 10.2 | 100 ± 17.3 |
| IIIn | COCH₂CH₂C₆H₅ | 2.0 | 111 ± 0.8 | 94 ± 2.5 | 111 ± 7.3 | 126 ± 6.1 |
| | | 20 | 101 ± 4.3 | 98 ± 4.7 | 106 ± 6.6 | 124 ± 13.4 |
| | | 200 | 54 ± 4.1 | 89 ± 5.2 | 76 ± 7.2 | 110 ± 10.5 |
| ^{a}Values are in μM and represent an average of three experiments. The variance for the relative viability (%) and relative SEAP activity (%) values was less than ±20%. Activity of P_{hTERT}-SEAP (H1299) and (hTERT-BJ1) | | | | | | |
| # cell growth was significantly different from that of the control; n = 3 or more, P < 0.01. Relative percentage of inhibition was not compared with that of the control, P < 0.01, mean ± S.E., n = 4. Values are the | | | | | | |
| # mean percent activity at the indicated concentration, and include standard errors. | | | | | | |

^{b}Non-small-cell lung cancer cells H1299.
^{c}The hTERT immortalized hTERT-BJ1 was purchased from BD Biosciences Clontech.

TABLE 12
Effects of Anthraquinones on the CMV Promoter Activity

P_{CMV}-SEAP(hTERT-BJ1)^{b}

| | Concn^{a} | Relative | Relative SEAP |
|---|---|---|---|
| Compd. | (μM) | viability (%) | activity (%) |
| IIf | 2.2 | 108 ± 9 | 103 ± 8 |
| | 22 | 109 ± 4 | 102 ± 10 |
| | 220 | 48 ± 6 | 122 ± 8 |
| IIj | 2.0 | 116 ± 13 | 103 ± 15 |
| | 22 | 67 ± 18 | 102 ± 8 |
| | 220 | 52 ± 7 | 100 ± 8 |
| IIn | 2.2 | 107 ± 10 | 112 ± 15 |
| | 22 | 118 ± 9 | 105 ± 16 |
| | 220 | 78 ± 18 | 120 ± 14 |
| IIIa | 2.8 | 114 ± 16 | 101 ± 6 |
| | 28 | 110 ± 12 | 112 ± 6 |
| | 280 | 94 ± 21 | 138 ± 12 |
| IIId | 2.4 | 115 ± 7 | 102 ± 10 |
| | 24 | 104 ± 8 | 114 ± 18 |
| | 240 | 103 ± 11 | 141 ± 11 |
| IIIi | 1.8 | 105 ± 8 | 99 ± 15 |
| | 18 | 105 ± 7 | 111 ± 11 |
| | 180 | 108 ± 15 | 121 ± 17 |
| ^{a}Values are in μM and represent an average of three experiments. The variance for the relative viability (%) and relative SEAP activity (%) values was less than ±20%. Activity of P_{CMV}-SEAP (hTERT-BJ1) cell growth was significantly different from | | | |
| # that of the control; n = 3 or more, P < 0.01. Relative percentage of inhibition was not compared with that of the control, P < 0.01, mean ± S.E., n = 4. Values are the mean percent activity at the indicated concentration and include standard errors. | | | |

^{b}CMV (cytomegalovirus); SEAP (secreted alkaline phosphatase).

TABLE 13
Effects of Symmetrical 1,4-Diamidoanthraquinones
(VI₁₋₃₇) on Activating hTERT Expression

| | | | P_{hTERT}-SEAP (hTERT-BJ1)^{b} | | |
|---|---|---|---|---|---|
| | | Conc. | Relative MTT | Relative SEAP | SEAP/ |
| No. | R | (μM)^{a} | viability (%) | activity (%) | vibility |
| VI₁ | CH₂Cl | 2.5 | 101 ± 2.7 | 123 ± 16.8 | 1.23 |
| | | 25 | 84 ± 6.3 | 129 ± 17.7 | 1.53 |
| | | 255 | 44 ± 4.7 | 121 ± 10.9 | 2.76 |
| VI₇ | (CH₂)₂Cl | 2.3 | 102 ± 4.1 | 36 ± 12.1 | 0.36 |
| | | 23 | 82 ± 8.2 | 40 ± 10.8 | 0.48 |
| | | 238 | 60 ± 2.5 | 14 ± 11.8 | 0.23 |
| VI₉ | CH(Cl)CH₃ | 2.3 | 94 ± 10.4 | 91 ± 10.8 | 0.97 |
| | | 23 | 68 ± 8.2 | 101 ± 8.4 | 1.49 |
| | | 238 | 48 ± 2.3 | 81 ± 8.5 | 1.68 |
| VI₃₃ | CH₂N(CH₂CH₃)₂ | 2.1 | 105 ± 2.8 | 111 ± 24.4 | 1.06 |
| | | 21 | 86 ± 3.5 | 120 ± 25.6 | 1.40 |
| | | 215 | 32 ± 13.1 | 83 ± 19.0 | 2.61 |
| VI₃₄ | (CH₂)₂N(CH₂CH₃)₂ | 2.0 | 83 ± 9.2 | 2 ± 11.1 | 0.03 |
| | | 20 | 15 ± 3.9 | (−2) ± 11.2 | −0.13 |
| | | 203 | 6 ± 3.9 | (−2) ± 6.5 | −0.37 |
| VI₃₅ | CH(CH₃)N(CH₂CH₂)₂ | 2.0 | 97 ± 3.9 | 40 ± 14.5 | 0.41 |
| | | 20 | 52 ± 7.2 | 49 ± 9.3 | 0.94 |
| | | 203 | 36 ± 5.8 | 22 ± 6.6 | 0.61 |
| VI₃₆ | CH(CH₂)NCH₂CH(CH₂)₂ | 2.0 | 103 ± 6.4 | 91 ± 19.4 | 0.88 |
| | | 20 | 92 ± 11.4 | 95 ± 14.2 | 1.03 |
| | | 204 | 104 ± 17.4 | 65 ± 15.7 | 0.63 |
| VI₃₇ | (CH₂)₂NCH₂CH(CH₂)₂ | 2.0 | 95 ± 2.5 | 97 ± 13.8 | 1.02 |
| | | 20 | 84 ± 1.5 | 124 ± 22.8 | 1.48 |
| | | 204 | 35 ± 3.7 | 105 ± 17.7 | 3.01 |
| VI₃ | CH₃ | 3.1 | 117 ± 3.0 | 77 ± 12.4 | 0.66 |
| | | 31 | 95 ± 11.9 | 83 ± 10.0 | 0.87 |
| | | 310 | 49 ± 8.9 | 59 ± 15.2 | 1.20 |
| VI₂₁ | cyclopropane | 2.6 | 103 ± 6.8 | 64 ± 9.5 | 0.62 |
| | | 26 | 82 ± 5.9 | 54 ± 8.8 | 0.66 |
| | | 267 | 58 ± 4.1 | 43 ± 8.3 | 0.74 |
| VI₂₀ | cyclopentane | 2.3 | 106 ± 2.3 | 85 ± 7.7 | 0.81 |
| | | 23 | 103 ± 4.4 | 93 ± 18.5 | 0.90 |
| | | 233 | 93 ± 5.7 | 63 ± 36.5 | 0.67 |
| VI₁₇ | cyclohexane | 2.1 | 102 ± 3.0 | 65 ± 16.0 | 0.64 |
| | | 21 | 97 ± 2.4 | 42 ± 24.3 | 0.44 |
| | | 218 | 83 ± 4.2 | 56 ± 16.3 | 0.68 |
| VI₁₉ | (CH₂)₂CH(CH₂)₄ | 2.0 | 107 ± 3.6 | 90 ± 9.2 | 0.84 |
| | | 20 | 101 ± 4.7 | 87 ± 5.5 | 0.87 |
| | | 205 | 100 ± 5.8 | 87 ± 8.0 | 0.87 |
| VI₂₂ | 2-SC(CH)₃ | 2.1 | 118 ± 10.6 | 94 ± 20.2 | 0.80 |
| | | 21 | 99 ± 8.2 | 92 ± 17.4 | 0.93 |
| | | 218 | 84 ± 6.9 | 90 ± 22.7 | 1.07 |
| VI₂₄ | 2-OC(CH)₃ | 2.3 | 91 ± 9.5 | 39 ± 7.8 | 0.43 |
| | | 23 | 69 ± 10.7 | 45 ± 11.0 | 0.65 |
| | | 234 | 72 ± 11.3 | 37 ± 15.0 | 0.51 |
| VI₂₅ | CH₂-2-SC(CH)₃ | 2.0 | 116 ± 7.5 | 53 ± 17.4 | 0.45 |
| | | 20 | 105 ± 4.4 | 40 ± 12.7 | 0.38 |
| | | 205 | 76 ± 6.5 | 14 ± 24.1 | 0.19 |
| VI₄ | C₆H₅ | 2.2 | 95 ± 4.9 | (−4) ± 25.7 | −0.04 |
| | | 22 | 69 ± 3.2 | (−10) ± 26.8 | −0.14 |
| | | 224 | 38 ± 2.6 | (−41) ± 26.8 | −1.10 |
| VI₆ | 3-CH₃C₆H₄ | 2.1 | 110 ± 2.6 | 97 ± 17.1 | 0.88 |
| | | 21 | 109 ± 4.6 | 78 ± 7.1 | 0.71 |
| | | 210 | 101 ± 6.0 | 70 ± 5.8 | 0.69 |
| VI₁₀ | 2-FC₆H₄ | 2.0 | 106 ± 6.5 | 95 ± 8.9 | 0.90 |
| | | 20 | 105 ± 8.5 | 96 ± 9.8 | 0.96 |
| | | 207 | 86 ± 7.3 | 79 ± 7.4 | 0.92 |
| VI₁₂ | 3-FC₆H₄ | 2.0 | 102 ± 1.5 | 107 ± 11.9 | 1.05 |
| | | 20 | 97 ± 2.9 | 108 ± 15.5 | 1.11 |
| | | 207 | 85 ± 2.6 | 95 ± 13.9 | 1.12 |
| VI₃₀ | 4-FC₆H₄ | 2.0 | 103 ± 9.0 | 104 ± 16.3 | 1.01 |
| | | 20 | 107 ± 3.1 | 101 ± 27.6 | 0.95 |
| | | 207 | 83 ± 5.3 | 100 ± 14.5 | 1.20 |
| VI₂ | 2-ClC₆H₄ | 1.9 | 116 ± 7.7 | 110 ± 20.1 | 0.95 |
| | | 19 | 109 ± 2.2 | 96 ± 33.4 | 0.88 |
| | | 194 | 95 ± 2.0 | 95 ± 36.6 | 1.00 |
| VI₅ | 3-ClC₆H₄ | 1.9 | 99 ± 9.8 | 98 ± 10.1 | 0.99 |
| | | 19 | 90 ± 1.9 | 105 ± 8.8 | 1.17 |
| | | 194 | 60 ± 2.0 | 89 ± 10.1 | 1.48 |
| VI₁₆ | 4-ClC₆H₄ | 1.9 | 111 ± 0.8 | 116 ± 28.6 | 1.05 |
| | | 19 | 103 ± 5.9 | 112 ± 21.9 | 1.09 |
| | | 194 | 99 ± 5.2 | 152 ± 39.7 | 1.54 |
| VI₁₁ | 2-NO₂C₆H₄ | 1.8 | 110 ± 4.1 | 102 ± 33.4 | 0.92 |
| | | 18 | 107 ± 6.3 | 122 ± 19.5 | 1.14 |
| | | 186 | 99 ± 3.2 | 114 ± 28.1 | 1.15 |
| VI₃₁ | 2-CF₃C₆H₄ | 1.7 | 98 ± 4.4 | 100 ± 15.1 | 1.02 |
| | | 17 | 100 ± 3.4 | 90 ± 16.3 | 0.90 |
| | | 171 | 89 ± 3.6 | 103 ± 17.6 | 1.16 |
| VI₂₉ | 2,3-(CF₃)₂C₆H₃ | 1.3 | 107 ± 6.4 | 16 ± 31.8 | 0.15 |
| | | 13 | 85 ± 4.8 | 24 ± 21.5 | 0.28 |
| | | 139 | 56 ± 4.6 | 26 ± 36.9 | 0.47 |
| VI₁₈ | 2,4-F₂C₆H₃ | 1.9 | 104 ± 5.6 | 48 ± 21.7 | 0.46 |
| | | 19 | 101 ± 4.4 | 51 ± 18.5 | 0.50 |
| | | 192 | 103 ± 6.1 | 48 ± 23.1 | 0.46 |
| VI₈ | 2,4-Cl₂C₆H₃ | 1.7 | 102 ± 3.4 | 33 ± 11.2 | 0.32 |
| | | 17 | 98 ± 7.2 | 25 ± 17.2 | 0.26 |
| | | 171 | 76 ± 4.8 | 39 ± 12.8 | 0.52 |
| VI₁₃ | 2,4,6-Cl₃C₆H₂ | 1.5 | 98 ± 8.5 | 31 ± 23.0 | 0.32 |
| | | 15 | 63 ± 7.0 | 12 ± 12.5 | 0.19 |
| | | 153 | 40 ± 22.2 | 4 ± 15 | 0.09 |
| VI₁₄ | 2,3,6-F₃C₆H₂ | 1.8 | 102 ± 3.3 | 35 ± 14.1 | 0.35 |
| | | 18 | 90 ± 6.0 | 22 ± 24.4 | 0.25 |
| | | 180 | 70 ± 6.8 | 31 ± 7.9 | 0.44 |
| VI₁₅ | 2,4,5-F₃C₆H₂ | 1.6 | 104 ± 4.5 | 117 ± 14.2 | 1.12 |
| | | 16 | 89 ± 5.0 | 114 ± 20.4 | 1.29 |
| | | 161 | 80 ± 3.8 | 115 ± 30.8 | 1.44 |
| VI₂₃ | 2,3-Cl₂-5-FC₆H₂ | 1.6 | 111 ± 6.8 | 113 ± 16.0 | 1.02 |
| | | 16 | 110 ± 7.1 | 131 ± 19.1 | 1.20 |
| | | 161 | 101 ± 6.6 | 106 ± 17.3 | 1.05 |
| VI₂₇ | CH(CH₂)CHC₆H₅ | 1.8 | 92 ± 16.9 | 92 ± 16.9 | 0.89 |
| | | 18 | 97 ± 18.2 | 97 ± 18.2 | 0.99 |
| | | 189 | 104 ± 10.7 | 102 ± 16.6 | 0.98 |
| VI₂₈ | CH₂SC₆H₅ | 1.8 | 115 ± 4.8 | 14 ± 14.7 | 0.12 |
| | | 18 | 105 ± 6.7 | 23 ± 9.5 | 0.21 |
| | | 185 | 60 ± 2.3 | 7 ± 9.6 | 0.12 |
| VI₃₂ | CH₂-4-FC₆H₄ | 1.9 | 107 ± 7.2 | 90 ± 18.4 | 0.84 |
| | | 19 | 100 ± 6.3 | 102 ± 20.0 | 1.02 |
| | | 195 | 96 ± 4.1 | 97 ± 11.6 | 1.01 |
| VI₂₆ | 2,5-dimethylfuran | 2.0 | 101 ± 6.2 | 84 ± 13.3 | 0.83 |
| | | 20 | 101 ± 5.8 | 79 ± 14.7 | 0.78 |
| | | 207 | 93 ± 7.1 | 81 ± 16.1 | 0.88 |
| ^{a}Values are in μM and represent an average of three experiments. The variance for the relative viability (%) and relative SEAP activity (%) | | | | | |
| # values was less than ±20%. Activity of P_{hTERT}-SEAP (hTERT-BJ1) cell growth was significantly different | | | | | |
| # from that of the control; n = 3 or more, P < 0.05. Relative percentage of inhibition was not compared with that of the control, P < 0.01, | | | | | |
| # mean ± S.E., n = 4. Values are mean percent activity at the indicated concentration, and standard errors. | | | | | |
| ^{b}The hTERT immortalized hTERT-BJ1 was purchased from BD Biosciences Clontech. | | | | | |
| Note: | | | | | |
| The results in this column are shown as means ± SE of experiments repeated five times. The different symbols qualify as in any concentration of treatment: | | | | | |
| # Relative Cell Viability > 80%, Relative SEAP activity > 100% and P value below 0.05 analyzed | | | | | |
| # with Two-tail T-test. The ratio of relative cell viability under relative SEAP activity is over 1.2. All of SEAP data are shown as the result that drug-self | | | | | |
| # interference has been subtracted. | | | | | |

TABLE 14
Effects of Symmetrical 1,4-diamidoanthraquinones
(VI₁₋₃₇) on Repressing hTERT Expression

| | | P_{hTERT}-SEAP(hTERT-H1299)^{b} | | | |
|---|---|---|---|---|---|
| | | Conc | Relative MTT | Relative SEAP | SEAP/ |
| No. | R | (μM)^{a} | viability (%) | activity (%) | vibility |
| | | 2.5 | 104 ± 5.5 | 102 ± 5.3 | 0.98 |
| VI₁ | CH₂Cl | 25 | 96 ± 3.8 | 110 ± 8.8 | 1.14 |
| | | 255 | 21 ± 1.5 | 97 ± 7.1 | 4.66 |
| VI₇ | (CH₂)₂Cl | 2.3 | 99 ± 6.0 | 87 ± 2.3 | 0.88 |
| | | 23 | 68 ± 2.4 | 87 ± 3.1 | 1.28 |
| | | 238 | 30 ± 3.8 | 68 ± 5.3 | 2.30 |
| VI₉ | CH(Cl)CH₃ | 2.3 | 111 ± 5.5 | 106 ± 5.5 | 0.96 |
| | | 23 | 77 ± 3.4 | 109 ± 1.0 | 1.40 |
| | | 238 | 11 ± 1.4 | 85 ± 5.3 | 7.82 |
| VI₃₃ | CH₂N(CH₂CH₃)₂ | 2.1 | 105 ± 4.9 | 98 ± 3.3 | 0.94 |
| | | 21 | 87 ± 3.9 | 86 ± 7.4 | 0.99 |
| | | 215 | 44 ± 2.7 | 48 ± 2.3 | 1.11 |
| VI₃₄ | (CH₂)₂N(CH₂CH₃)₂ | 2.0 | 92 ± 6.1 | 73 ± 2.7 | 0.79 |
| | | 20 | 11 ± 2.5 | 53 ± 2.7 | 4.63 |
| | | 203 | 1 ± 2.4 | 39 ± 0.8 | 44.24 |
| VI₃₅ | CH(CH₃)N(CH₂CH₂)₂ | 2.0 | 96 ± 7.9 | 96 ± 14.3 | 1.00 |
| | | 20 | 77 ± 7.2 | 104 ± 6.0 | 1.35 |
| | | 203 | 75 ± 4.6 | 73 ± 11.7 | 0.98 |
| VI₃₆ | CH(CH₂)NCH₂CH(CH₂)₂ | 2.0 | 106 ± 4.1 | 123 ± 12.4 | 1.16 |
| | | 20 | 64 ± 5.1 | 112 ± 11.5 | 1.76 |
| | | 204 | 30 ± 4.7 | 80 ± 14.5 | 2.71 |
| VI₃₇ | (CH₂)₂NCH₂CH(CH₂)₂ | 2.0 | 108 ± 7.8 | 95 ± 4.2 | 0.88 |
| | | 20 | 83 ± 1.6 | 91 ± 8.9 | 1.09 |
| | | 204 | 67 ± 9.8 | 52 ± 4.4 | 0.77 |
| VI₃ | CH₃ | 3.1 | 109 ± 3.5 | 85 ± 2.3 | 0.78 |
| | | 31 | 108 ± 3.6 | 90 ± 3.3 | 0.83 |
| | | 310 | 37 ± 1.3 | 80 ± 3.1 | 2.16 |
| VI₂₁ | cyclopropane | 2.6 | 95 ± 4.2 | 108 ± 4.9 | 1.14 |
| | | 26 | 88 ± 4.2 | 106 ± 7.3 | 1.21 |
| | | 267 | 41 ± 4.6 | 92 ± 5.9 | 2.23 |
| VI₂₀ | cyclopentane | 2.3 | 107 ± 6.1 | 119 ± 8.5 | 1.11 |
| | | 23 | 103 ± 6.1 | 118 ± 9.2 | 1.15 |
| | | 233 | 92 ± 8.6 | 113 ± 8.9 | 1.23 |
| VI₁₇ | cyclohexane | 2.1 | 104 ± 6.4 | 104 ± 1.8 | 1.00 |
| | | 21 | 96 ± 5.8 | 100 ± 6.2 | 1.05 |
| | | 218 | 67 ± 1.5 | 93 ± 2.5 | 1.38 |
| VI₁₉ | (CH₂)₂CH(CH₂)₄ | 2.0 | 105 ± 2.1 | 111 ± 9.4 | 1.06 |
| | | 20 | 106 ± 4.7 | 116 ± 8.0 | 1.09 |
| | | 205 | 95 ± 1.3 | 109 ± 9.2 | 1.15 |
| VI₂₂ | 2-SC(CH)₃ | 2.1 | 108 ± 3.7 | 100 ± 3.1 | 0.93 |
| | | 21 | 107 ± 8.5 | 105 ± 5.9 | 0.98 |
| | | 218 | 84 ± 4.3 | 97 ± 4.9 | 1.16 |
| VI₂₄ | 2-OC(CH)₃ | 2.3 | 101 ± 6.0 | 99 ± 7.8 | 0.99 |
| | | 23 | 75 ± 3.9 | 97 ± 4.6 | 1.30 |
| | | 234 | 32 ± 5.1 | 90 ± 2.9 | 2.83 |
| VI₂₅ | CH₂-2-SC(CH)₃ | 2.0 | 104 ± 8.0 | 111 ± 3.7 | 1.06 |
| | | 20 | 83 ± 6.6 | 115 ± 4.3 | 1.39 |
| | | 205 | 41 ± 4.0 | 102 ± 5.5 | 2.47 |
| VI₄ | C₆H₅ | 2.2 | 109 ± 6.0 | 81 ± 3.3 | 0.74 |
| | | 22 | 88 ± 6.2 | 84 ± 2.6 | 0.96 |
| | | 224 | 27 ± 1.8 | 80 ± 2.7 | 2.99 |
| VI₆ | 3-CH₃C₆H₄ | 2.1 | 101 ± 5.6 | 107 ± 4.6 | 1.06 |
| | | 21 | 97 ± 6.2 | 104 ± 4.0 | 1.08 |
| | | 210 | 83 ± 7.8 | 99 ± 9.1 | 1.20 |
| VI₁₀ | 2-FC₆H₄ | 2.0 | 116 ± 2.4 | 105 ± 7.8 | 0.91 |
| | | 20 | 104 ± 6.1 | 107 ± 11.9 | 1.03 |
| | | 207 | 77 ± 2.6 | 103 ± 14.4 | 1.34 |
| VI₁₂ | 3-FC₆H₄ | 2.0 | 105 ± 6.8 | 102 ± 2.9 | 0.98 |
| | | 20 | 96 ± 7.4 | 110 ± 6.5 | 1.14 |
| | | 207 | 66 ± 5.5 | 106 ± 9.4 | 1.59 |
| VI₃₀ | 4-FC₆H₄ | 2.0 | 110 ± 5.9 | 111 ± 3.6 | 1.01 |
| | | 20 | 103 ± 6.6 | 107 ± 4.8 | 1.04 |
| | | 207 | 89 ± 6.7 | 107 ± 9.0 | 1.20 |
| VI₂ | 2-ClC₆H₄ | 1.9 | 114 ± 5.4 | 108 ± 10.5 | 0.94 |
| | | 19 | 72 ± 3.6 | 105 ± 7.7 | 1.47 |
| | | 194 | 60 ± 7.9 | 100 ± 8.9 | 1.68 |
| VI₅ | 3-ClC₆H₄ | 1.9 | 95 ± 11.3 | 110 ± 4.9 | 1.17 |
| | | 19 | 102 ± 8.5 | 112 ± 6.1 | 1.10 |
| | | 194 | 59 ± 4.3 | 91 ± 3.0 | 1.55 |
| VI₁₆ | 4-ClC₆H₄ | 1.9 | 89 ± 5.5 | 109 ± 6.8 | 1.23 |
| | | 19 | 89 ± 9.5 | 118 ± 5.6 | 1.33 |
| | | 194 | 79 ± 5.6 | 116 ± 12.3 | 1.46 |
| VI₁₁ | 2-NO₂C₆H₄ | 1.8 | 104 ± 6.3 | 105 ± 4.3 | 1.00 |
| | | 18 | 101 ± 4.1 | 107 ± 7.9 | 1.06 |
| | | 186 | 86 ± 5.3 | 94 ± 8.0 | 1.10 |
| VI₃₁ | 2-CF₃C₆H₄ | 1.7 | 98 ± 7.8 | 110 ± 5.5 | 1.12 |
| | | 17 | 100 ± 8.9 | 114 ± 5.6 | 1.14 |
| | | 171 | 77 ± 6.7 | 96 ± 2.1 | 1.24 |
| VI₂₉ | 2,3-(CF₃)₂C₆H₃ | 1.3 | 99 ± 8.6 | 106 ± 6.7 | 1.07 |
| | | 13 | 79 ± 10.3 | 105 ± 7.9 | 1.32 |
| | | 139 | 37 ± 8.8 | 94 ± 10.2 | 2.56 |
| VI₁₈ | 2,4-F₂C₆H₃ | 1.9 | 87 ± 3.7 | 93 ± 5.6 | 1.06 |
| | | 19 | 85 ± 7.1 | 99 ± 5.4 | 1.17 |
| | | 192 | 69 ± 8.4 | 99 ± 1.8 | 1.44 |
| VI₈ | 2,4-Cl₂C₆H₃ | 1.7 | 106 ± 3.9 | 85 ± 7.9 | 0.80 |
| | | 17 | 95 ± 1.3 | 84 ± 7.1 | 0.89 |
| | | 171 | 83 ± 1.2 | 87 ± 2.6 | 1.06 |
| VI₁₃ | 2,4,6-Cl₃C₆H₂ | 1.5 | 113 ± 4.8 | 98 ± 4.5 | 0.87 |
| | | 15 | 107 ± 5.4 | 66 ± 2.4 | 0.61 |
| | | 153 | 20 ± 3.3 | 41 ± 3.9 | 0.03 |
| VI₁₄ | 2,3,6-F₃C₆H₂ | 1.8 | 111 ± 2.9 | 91 ± 5.2 | 0.82 |
| | | 18 | 89 ± 3.7 | 97 ± 4.1 | 1.09 |
| | | 180 | 55 ± 3.3 | 91 ± 3.3 | 1.64 |
| VI₁₅ | 2,4,5-F₃C₆H₂ | 1.6 | 90 ± 6.6 | 109 ± 10.8 | 1.20 |
| | | 16 | 81 ± 1.9 | 118 ± 10.3 | 1.45 |
| | | 161 | 60 ± 5.0 | 104 ± 8.2 | 1.73 |
| VI₂₃ | 2,3-Cl₂-5-FC₆H₂ | 1.6 | 98 ± 7.0 | 102 ± 4.1 | 1.04 |
| | | 16 | 103 ± 8.7 | 102 ± 5.1 | 0.99 |
| | | 161 | 87 ± 5.1 | 83 ± 4.0 | 0.95 |
| VI₂₇ | CH(CH₂)CHC₆H₅ | 1.8 | 101 ± 2.4 | 95 ± 7.7 | 0.94 |
| | | 18 | 100 ± 4.7 | 93 ± 3.2 | 0.93 |
| | | 189 | 94 ± 3.5. | 87 ± 3.6 | 0.92 |
| VI₂₈ | CH₂SC₆H₅ | 1.8 | 104 ± 5.6 | 93 ± 3.2 | 0.89 |
| | | 18 | 96 ± 4.3 | 92 ± 3.5 | 0.96 |
| | | 185 | 59 ± 3.8 | 80 ± 4.0 | 1.35 |
| VI₃₂ | CH₂-4-FC₆H₄ | 1.9 | 108 ± 8.8 | 109 ± 10.8 | 1.00 |
| | | 19 | 107 ± 7.5 | 109 ± 5.8 | 1.03 |
| | | 195 | 78 ± 8.0 | 109 ± 6.6 | 1.39 |
| VI₂₆ | 2,5-dimethylfuran | 2.0 | 87 ± 4.9 | 104 ± 10.8 | 1.20 |
| | | 20 | 71 ± 2.3 | 103 ± 10.0 | 1.46 |
| | | 207 | 47 ± 2.6 | 98 ± 10.5 | 2.10 |
| | Mitoxnatrone | 1.9 | 100 ± 5.6 | 81 ± 3.8 | 0.82 |
| | | 19 | 57 ± 4.3 | 66 ± 4.0 | 1.16 |
| | | 193 | 39 ± 3.2 | 47 ± 3.9 | 1.21 |
| ^{a}Values are in μM and represent an average of three experiments. The variance for the relative viability (%) and relative SEAP activity | | | | | |
| # (%) values was less than ±20%. Repression of P_{hTERT}-SEAP (hTERT-H1299) cell growth was significantly different | | | | | |
| # from that of the control; n = 3 or more, P < 0.05. Relative percentage of inhibition was not compared with that of the control, P < 0.01, | | | | | |
| # mean ± S.E., n = 4. Values are mean percent activity at the indicated concentration, and standard errors. | | | | | |

^{b}The hTERT cancer cell hTERT-H1299 was purchased from BD Biosciences Clontech.

1. A pharmaceutical compound according to Formula I, 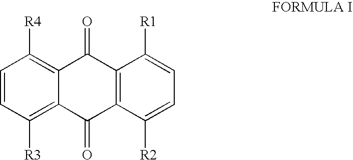
wherein R1, R2, R3 and R4 are selected from the group consisting of a straight or branched chain alkyl group having 1 to 6 carbons substitutes with one or more Ra groups, a benzyl group, a phenyl group which is substituted with one or two Rb groups, and a benzyl group which is substituted with one or two Rb groups;
wherein R1, R2, R3 and R4 are selected from the group consisting of halogen, —NO₂, —OCH₃, —OCH₂CH₃, —CH(CH₃)₂, —(CH₂)ₙOH, —(CH₂)ₙNH, —CH₂CH₂N(CH₃)₂, —CH₂CH₂N H(CH₂)₂OH, cyclopentane, 2,3-(CH₃)₂-cyclohexane, —S-Rc, —O—CO-Rd, —N-Re, —CO-Rf, —CONH-Rg; and
wherein Ra, Rb, Rc, Rd, Re, Rf, Rg are selected from the group consisting of a straight or branched chain alkyl group having 1 to 6 carbons, —NO₂, —OCH₃, —OCH₂CH₃, —CH(CH₃)₂, —(CH₂)ₙOH, —(CH₂)ₙNH, —CH₂CH₂N(CH₃)₂, —CH₂CH₂NH(CH₂)₂OH, cyclopentane, 2,3-(CH₃)₂-cyclohexane, —S—, —OCO—, —N—, —CO—, —CONH—.

2. The compound according to claim 1, wherein R1, R2, R3 and R4 represent a substituted phenyl, benzyl, ethylphenyl, cyclopentane, and 2,3-(CH₃)₂-cyclohexane groups selected from the group consisting of 2-CH₃C₆H₄, 3-CH₃C₆H₄, 4-CH₃C₆H₄, 2-OHC₆H₄, 3-OHC₆H₄, 4-OHC₆H₄, 2-CIC₆H₄, 3-CIC₆H₄, 4-CIC₆H₄, 2-NO₂C₆H₄, 3-NO₂C₆H₄, 4-NO₂C₆H₄, 2-NH₂C₆H₄, 3-NH₂C₆H₄, 4-NH₂C₆H₄, and 2,4-Cl₂C₆H₃.

3. The compound according to claim 1, wherein R1, R2, R3 and R4 represent a substituted alkyl group selected from the group consisting of CH₂Br, CH₂Cl, CH₂OH, C(CH₃)₃, (CH₂)₂OH, (CH₂)₃OH, (CH₂)₄OH, CH₂NH₂, (CH₂)₂NH₂, (CH₂)₃NH₂, (CH₂)₄NH₂, (CH₂)₅NH₂, CH₂N(CH₃)₂, (CH₂)₂N(CH₃)₂, (CH₂)₂NH(CH₂)₂OH(CH₂)₃NH(CH₂)₂OH, (CH₂)₂NHCH₂OH, (CH₂)₃NHCH₂OH, CH₂CH(CH₃)₂, CHCl₂, CH(CH₃)Cl, (CH₂)₂Cl, (CH₂)₃Cl, (CH₂)₃Br, (CH₂)₄Br, and (CH₂)₄Cl.

4. An anti-cancer drug, comprising, as an active ingredient, the pharmaceutical compound of claim 1.

5. A telomerase effect drug, comprising, as an active ingredient, the pharmaceutical compound of claim 1.

6. An anti-inflammatory drug, comprising, as an active ingredient, the pharmaceutical compound of claim 1.

7. An anti-oxidant drug, comprising, as an active ingredient, the pharmaceutical compound of claim 1.

8. An anti-psoriatic drug, comprising, as an active ingredient, the pharmaceutical compound of claim 1.

9. A stem cell and tissue engineering application, comprising, as an active ingredient, the pharmaceutical compound of claim 1.

10. A compound having the chemical structure of Formula I, 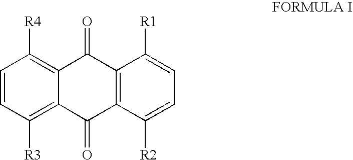
wherein R1, R2, R3 and R4 represent cyclopentane, cyclohexane, —C₆H₅, —CH₂C₆H₅, or —CH₂CH₂C₆H₅, group having one, two or three substituents which is selected from the group of halogen, OH, CH₃, OCH₃, NH₂, and NO₂.

11. A compound having the chemical structure of Formula I, 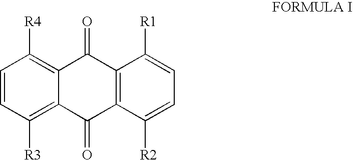
wherein R1, R2, R3 and R4 represent —S—, —O—CO—, —N—, —CO—, and —CONH—, consisting of a straight or branched chain alkyl group having 1 to 6 carbons, and CH₂Br, CH₂Cl, CH₂OH, C(CH₃)₃, (CH₂)₂OH, (CH₁₂)₃OH, (CH₂)₄OH, CH₂NH₂, (CH₂)₂NH₂, (CH₂)₃NH₂, (CH₂)₄NH₂, (CH₂)₅NH₂, CH₂N(CH₃)₂, (CH₂)₂N(CH₃)₂, (CH₂)₂NH(CH₂)₂OH, (CH₂)₃NH(CH₂)₂OH, (CH₂)₂NHCH₂OH, (CH₂)₃NHCH₂OH, CH₂CH(CH₃)₂, CHCl₂, CH(CH₃)Cl, (CH₂)₂Cl, (CH₂)₃Cl, (CH₂)₃Br, (CH₂)₄Br, and (CH₂)₄Cl.

12. A method for synthesis of bis-substituted anthraquinone compounds and salts thereof, comprising reacting 1,5-dichloroanthraquinone, anthrarufin, 1,8-dichloroanthraquinone, 1,5-diaminoanthraquinone or 1,8-diaminoanthraquinone with an appropriate acyl chlorides, thiols, or amines under appropriate conditions to give the bis-substituted anthraquinones according to Formula I 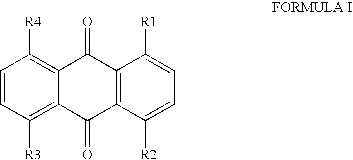
wherein R1, R2, R3 and R4 are selected from the group consisting of a straight chain alkyl group having 1 to 6 carbons which is optionally substituted with one or more R groups, a branched chain alkyl group having 1 to 6 carbons which is optically substituted with one or more R groups, cyclopentane, 2,3-(CH₃)₂-cyclohexane, —C₆H₅, —CH₂C₆H₅, or —CH₂CH₂C₆H₅, a phenyl which is substituted with one or more R groups, and a benzyl group which is optionally substituted with one or more R groups, and —CH₂CH₂C₆H₅ group which is optionally substituted with one or more R groups;
wherein R is selected from the group consisting of halogen, OH, CH₃, OCH₃, NH₂, and NO₂.

13. A method for anti-cancer treatment, comprising administering a therapeutically effective amount of a pharmaceutical compounds according to claim 11 or a pharmaceutically acceptable salt of said compound and optionally a pharmaceutical carrier to a patient in need of such treatment.

14. A method for treating abnormal, proliferation, comprising administering a therapeutically effective amount of a pharmaceutical compounds according to claim 11 or a pharmaceutically acceptable salt of said compound and optionally a pharmaceutical carrier to a patient in need of such treatment.

15. A method for enhancing an anti-oxidation affect, comprising administering a therapeutically effective amount of a pharmaceutical compounds according to claim 11 or a pharmaceutically acceptable salt of said compound and optionally a pharmaceutical carrier to a patient in need of such treatment.

16. A method for enhancing human telomerase activity, comprising administering a therapeutically effective amount of a pharmaceutical compounds according to claim 11 or a pharmaceutically acceptable salt of said compound and optionally a pharmaceutical carrier to a patient in need of such treatment.

17. A method for stem cell research, comprising administering a therapeutically effective amount of a pharmaceutical compounds according to claim 11 or a pharmaceutically acceptable salt of said compound and optionally a pharmaceutical carrier to a patient in need of such treatment.

18. A method for enhancing tissue engineering application, comprising administering a therapeutically effective amount of a pharmaceutical compounds according to claim 11 or a pharmaceutically acceptable salt of said compound and optionally a pharmaceutical carrier to a patient in need of such treatment.

19. An anti-cancer drug, comprising, as an active ingredient, the pharmaceutical compound of claim 11.

20. An anti-inflammatory drug, comprising, as an active ingredient, the pharmaceutical compound of claim 11.

21. An anti-oxidant drug, comprising, as an active ingredient, the pharmaceutical compound of claim 11.

22. An anti-psoriatic drug comprising, as an active ingredient, the pharmaceutical compound of claim 11.

23. Drug for telomerase activation or inhibition, comprising, as an active ingredient, the pharmaceutical compound of claim 11.

24. Drug for stem cell application, comprising, as an active ingredient, the pharmaceutical compound of claim 11.

25. Drug for tissue engineering, comprising, as an active ingredient, the pharmaceutical compound of claim 11.
